(19) United States
(12) Patent Application Publication
(10) Pub. No.: US 2025/0242008 A1
(43) Pub. Date: Jul. 31, 2025

(54) PFCSP-BASED IMMUNOGENS AND RELATED COMPOSITION AND METHODS

(71) Applicants: THE HOSPITAL FOR SICK CHILDREN, Toronto (CA); DEUTSCHES KREBSFORSCHUNGSZENTRUM STIFTUNG DES ÖFFENTLICHEN RECHTS, Heidelberg (DE); MAX-PLANCK-GESSELLSCHAFT ZUR FOERDERUNG DER WISSENSCHAFTEN E V., Muenchen (DE)

(72) Inventors: Hedda WARDEMANN, Heidelberg (DE); Rajagopal MURUGAN, Heidelberg (DE); Anna OBRAZTSOVA, Heidelberg (DE); Elena LEVASHINA, Berlin (DE); Giulia COSTA, Berlin (DE); Jean-philippe JULIEN, Toronto (CA); Katherine PRIETO, Toronto (CA); Elaine THAI, Toronto (CA)

(21) Appl. No.: 18/856,692

(22) PCT Filed: Apr. 13, 2023
(86) PCT No.: PCT/CA2023/050505

§ 371 Date: Oct. 14, 2024

Related U.S. Application Data
(60) Provisional application No. 63/330,903, filed on Apr. 14, 2022.

(51) Int. Cl. A61K 39/015; A61K 39/00; A61K 39/385; A61P 33/06; C07K 14/445; C07K 16/20

(52) U.S. Cl. CPC A61K 39/015; A61K 39/385; A61P 33/06; C07K 14/445; C07K 16/205; A61K 2039/545; A61K 2039/6037; A61K 2039/627; C07K 2319/91

(57) ABSTRACT

Described herein is malarial immnunogen or a variant thereof comprising at least a portion of the wild-type PfCSP amino acid sequence lacking a KQ motif. In aspects, the malarial immunogen is lacking a KQP motif. For example, the immnunogens described herein, in aspects, exclude the C-terminal domain of PfCSP. In other aspects, the immunogens described herein specifically exclude a KQ or KQP motif. In aspects the immunogens described herein exclude an N-terminal KQ or KQP motif, which is part of the N-terminal junction region in PfCSP.

### FIELD

The present invention relates to immunogens. In particular, the present invention relates to malarial immunogens based on PfCSP antigens, and related compositions and methods.

### BACKGROUND

Plasmodium falciparum (Pf) is the parasite that accounts for most malaria fatalities globally, but a highly efficacious pre-erythrocytic subunit vaccine remains elusive. Previous studies, such as Foquet, L. et al. (J. Clin. Invest. 124, 140-144, 2014), White, M. T. et al. (PLoS One 8, e61395, 2013), RTSS Clinical Trials Partnership (Lancet 386, 31-45, 2015), and Sumitani, M. et al. (Insect Mol. Biol. 22, 41-51, 2013), have shown that protective antibodies against malaria exist that recognize the NANP repeat of the Circumsporozoite protein (CSP) on the surface of the Pf sporozoites.

The RTS,S/AS01 malaria subunit vaccine (GSK) contains 18.5 CSP NANP repeats and the complete C-terminal domain (C-CSP), displayed on a virus-like particle composed of Hepatitis B surface antigen building blocks. RTS,S/AS01 protected approximately 50% of vaccinated individuals in a recent phase Ill trial in Africa, but its efficacy waned rapidly (Agnandji et al., 2011; RTSS Clinical Trials Partnership, 2015).

U.S. Patent Application Publication Nos. 2013/0259890 and 2016/0038580 describe a nucleotide sequence and other constructs used for expression of recombinant P. falciparum circumsporozoite proteins in bacterial cells such as E. coli. Processes for producing a soluble recombinant P. falciparum CSP from E. coli are described. Methods to produce a human-grade, highly immunogenic anti-malaria vaccine based on CSP are shown. The recombinant P. falciparum circumsporozoite protein by itself or in combination with other malaria antigens or adjuvants are described as forming the basis of an effective malaria vaccine.

International Patent Application Publication No. 2018/193063 describes a fragment of Plasmodium circumsporozoite protein, for example for use in a malaria vaccine, nucleic acids encoding a fragment of Plasmodium CSP, compositions comprising a fragment of Plasmodium CSP, and antibodies binding to a fragment of Plasmodium CSP. The antibodies bind specifically to P. falciparum sporozoites and may be used in the treatment and/or prevention of malaria.

International Patent Application Publication No. 2012/154199 describes nucleotide sequences and other constructs used for expression of recombinant P. falciparum circumsporozoite proteins in bacterial cells such as E. coli. Processes are described for producing a soluble recombinant P. falciparum CSP from E. coli. Methods to produce a human-grade, highly immunogenic anti-malaria vaccine based on CSP are described.

A need exists for the development of an effective malaria vaccine as well as alternative vaccine platforms and related vaccines, compositions, and methods.

### SUMMARY

In accordance with an aspect, there is provided a malarial immunogen or a variant thereof comprising at least a portion of the wild-type PfCSP amino acid sequence lacking a KQ motif.

In an aspect, the malarial immunogen lacks a KQP motif.

In an aspect, the malarial immunogen lacks an N-terminal ETG motif.

In an aspect, the malarial immunogen comprises the following motifs:
NPDPₐ; PDPNₐ; DPNPₐ; or PNPDₐ;
NANP_{b}; ANPN_{b}; NPNA_{b}; or PNAN_{b};
NVDP_{c}; VDPN_{c}; DPNV_{c}; or PNVD_{c}; and
NANP_{d}; ANPN_{d}; NPNA_{d}; or PNAN_{d}.
wherein a, b, c, and d are each independently 0 or greater and wherein a+b+c+d is at least 2.

In an aspect, a, b, c, d or any combination thereof are each independently at least about 1.

In an aspect, a, b, c, d or any combination thereof are each independently from about 1 to about 40.

In an aspect, a, b, c, and d are each independently from about 1 to about 100, such as from about 1, about 2, about 3, about 4, about 5, about 6, about 7, about 8, about 9, about 10, about 11, about 12, about 13, about 14, about 15, about 16, about 17, about 18, about 19, or about 20 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 60, about 70, about 75, about 80, about 90, or about 100, such as from about 1 to about 40, or about 1 to about 20, or from 1 to about 10.

In an aspect, a is 1.

In an aspect, b is 1.

In an aspect, c is 3.

In an aspect, d is 5.

In an aspect, d is 18.5.

In an aspect, when a, b, c, and/or d are greater than 1 such that the respective motif is at least partially repeated, the repeated motifs are each independently contiguous.

In an aspect, when a, b, c, and/or d are greater than 1 such that the respective motif is at least partially repeated, the repeated motifs are each independently non-contiguous.

In an aspect, the motifs are in the order NPDPₐ-NANP_{b}-NVDP_{c}-NANP_{d}.

In an aspect, the malarial immunogen further comprises an N-terminal ADG or PADG motif, wherein the ADG or PADG motif optionally repeats alone or in combination with at least one of the NPDPₐ, NANP_{b}, NVDP_{c}, and NANP_{d} motifs.

In an aspect, the variant comprises at least about 70%, 75%, 80%, 85%, 90%, 95%, 97%, or 99% sequence identity to the wild-type PfCSP amino acid sequence lacking the KQ motif.

In an aspect, the malarial immunogen is fused directly or indirectly to a nanocage monomer peptide.

In an aspect, the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof.

In an aspect, the nanocage monomer is provided as two or more self-assembling subunits.

In an aspect, the nanocage monomer peptide is from Helicobacter pylori.

In an aspect, the nanocage monomer peptide is not human.

In an aspect, the nanocage monomer peptide is modified to reduce an anti-nanocage monomer peptide immune response.

In an aspect, the nanocage monomer peptide is modified to enhance the antigen immune response.

In an aspect, the nanocage monomer peptide is at least partially or fully covered.

In an aspect, the nanocage monomer peptide is at least partially glycan covered.

In an aspect, the nanocage monomer peptide is fully glycan covered.

In an aspect, the nanocage monomer comprises at least one NXT and/or NXS glycosylation motif.

In an aspect, the nanocage monomer is glycosylated at N79 and/or N99.

In an aspect, the nanocage monomer is glycosylated.

In an aspect, the nanocage monomer is glycosylated with high-mannose glycans.

In an aspect, a plurality of the nanocage monomer peptides self-assemble into a nanocage.

In an aspect, the immunogenic peptide decorates the interior and/or exterior surface of the nanocage.

In an aspect, the malarial immunogen further comprises a peptide that provides exogenous T cell help and/or a peptide that provides autologous T cell help.

In an aspect, the peptide that provides exogenous T cell help comprises a PADRE peptide and/or a peptide derived from a pathogenic molecule, such as a tetanus toxoid peptide.

In an aspect, the PADRE peptide comprises the amino acid sequence AKFVAAWTLKAAA, or a functional variant thereof having at least 70% sequence identity thereto or a fragment of either thereof.

In an aspect, the peptide that provides autologous T cell help comprises a PfCSP T cell peptide epitope.

In an aspect, the peptide that provides exogenous T cell help and/or the peptide that provides autologous T cell help independently decorates the interior and/or exterior surface of the assembled nanocage.

In an aspect, the malarial immunogen further comprises a linker between any one or more of the motifs, the nanocage monomer, and any further peptides, such as the peptide that provides exogenous T cell help and/or the peptide that provide autologous T cell help.

In an aspect, the linker is a GGS linker.

In an aspect, the linker comprises the amino acid sequence:

GGS;
GGGGSGGSGGSGGS;
and/or
GGGGGSGGSGGSGGS.

In an aspect, the malarial immuno en comprises or consists of the sequence:

ADG-NPDP-NANPNVDP3-NANP5-Hpferr-PADRE;
ADG-NPDP-NANPNVDP3-NANP18-Hpferr-PADRE;
ADG-NPDP-NANPNVDP3-NANP18.5-Hpferr-PADRE;
ADG-NPDP-NANPNVDP3-NANP5-LS-PADRE;
ADG-NPDP-NANPNVDP3-NANP18-LS-PADRE;
and/or
ADG-NPDP-NANPNVDP3-NANP18.5-LS-PADRE.

In an aspect, the malarial immunogen comprises any one of the following amino acid sequences:

203:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHNFTGLTQIF
QKAYEHEQHISNSTNNIVDHAIKSKDHATFNFLQWYVAEQHEEEVLFKDI
LDKIELIGNENHGLYLADQYVKGIAKSRKSGGSASAKFVAAWTLKAAA
205:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHNFTGLTQ
IFQKAYEHEQHISNSTNNIVDHAIKSKDHATFNFLQWYVAEQHEEEVLFK
DILDKIELIGNENHGLYLADQYVKGIAKSRKSGGSASAKFVAAWTLKAAA
206:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFK
DILDKIELIGNENHGLYLADQYVKGIAKSRKSGGSASAKFVAAWTLKAAA
207:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHNFTGLTQIF
QKAYEHEQHISNSTNNIVDHAIKSKDHATFNFLQWYVAEQHEEEVLFKDI
LDKIELIGNENHGLYLADQYVKGIAKSRKS
208:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHNFTGLTQ
IFQKAYEHEQHISNSTNNIVDHAIKSKDHATFNFLQWYVAEQHEEEVLFK
DILDKIELIGNENHGLYLADQYVKGIAKSRKS
209:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDI
LDKIELIGNENHGLYLADQYVKGIAKSRKS
210:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFK
DILDKIELIGNENHGLYLADQYVKGIAKSRKS
211:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDI
LDKIELIGNENHGLYLADQYVKGIAKSRKSGGSASAKFVAAWTLKAAA
212:
ADGNPDPNANPNVDPNANPNVDPNANPNVDPNANPNANPNANPNANPNAN
PGGSGGSGGSGGSGGSDIIKLLNEQVNKEMQSSNLYMSMSSWCYTHSLDG
AGLFLFDHAAEEYEHAKKLIIFLNENNVPVQLTSISAPEHKFEGLTQIFQ
KAYEHEQHISESINNIVDHAIKSKDHATFNFLQWYVAEQHEEEVLFKDIL
DKIELIGNENHGLYLADQYVKGIAKSRKSGGSASAKFVAAWTLKAAA
213:
ADGNPDPNANPNVDPNANPNVDPNANPNVDPNANPNANPNANPNANPNAN
PGGSGGSGGSGGSGGSDIIKLLNEQVNKEMQSSNLYMSMSSWCYTHSLDG
AGLFLFDHAAEEYEHAKKLIIFLNENNVPVQLTSISAPEHNFTGLTQIFQ
KAYEHEQHISNSTNNIVDHAIKSKDHATFNFLQWYVAEQHEEEVLFKDIL
DKIELIGNENHGLYLADQYVKGIAKSRKSGGSASAKFVAAWTLKAAA

or a functional variant having at least 70% sequence identity thereto, or a functional fragment of either thereof.

In accordance with an aspect, there is provided a nucleic acid molecule encoding the malarial immunogen described herein.

In accordance with an aspect, there is provided a vector comprising the nucleic acid molecule described herein.

In accordance with an aspect, there is provided a host cell comprising the vector of claim 40 and producing the malarial immunogen described herein.

In accordance with an aspect, there is provided a vaccine comprising the malarial immunogen described herein.

In an aspect, the vaccine further comprises an adjuvant.

In accordance with an aspect, there is provided an antibody that binds to the malarial immunogen described herein.

In accordance with an aspect, there is provided a method of treating and/or preventing malaria, comprising administering the immunogen, nucleotide, vector, cell, or vaccine described herein.

In accordance with an aspect, there is provided a use of the immunogen, nucleotide, vector, cell, or vaccine described herein for treating and/or preventing malaria.

In an aspect, the immunogen, nucleotide, vector, cell, or vaccine described herein is for use in treating and/or preventing malaria.

In an aspect, the preventative and/or treatment effect is boostable.

In an aspect, the preventative and/or treatment effect persists for at least about 6 months or more, such as about 9 months or more, about 12 months or more, about 1, about 2, about 3, about 4, about 5, about 6, about 7, about 8, about 9, or about 10 years or more.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1. Strong recruitment of IGHV3-33 and IGHV3-15 expressing B cells in response to immunization with KQP-containing immunogens. Mice were immunized subcutaneously (s.c.) with immunogens containing N-junction amino acids and NANP units repeated 5 or 18 times in either Hp-ferritin or lumazine synthase nanoparticles (126, 127, or 128 or 129) adjuvanted with SAS on days 1, 28 and 70 and sacrificed on day 77. Cells from lymph nodes and bone marrow were collected. IGHV gene segment usage. A) Among bone marrow plasma cells (BM PC), lymph node plasma cells (LN PC) and germinal center B cells (LN GCB). B) Among CSP+ and CSP− germinal center cells (GCB CSP+ and GCB CSP−) and pooled plasma cells from lymph nodes and bone marrow (PC) as identified by flow cytometry. Dots represent individual animals, only populations with >10 cells per animal were included in the analysis. N=2 experiments, the total number of sequences is 11430 (476 sequences per animal on average).

FIG. 2. Dominant pairing of IGHV3-15 with IGLV3-10. IGK/LV gene segment usage among CSP+ germinal center B cells expressing VH3-15 antibodies from FIG. 1.

FIG. 3. VH3-15/VL3-10 mAbs are KQPA-peptide specific with up to nanomolar affinity. A. ELISA reactivity of VH3-15/VL3-10 (circles) and VH3-33/VK1-5 (triangles) mAbs to the KQPA peptide when the peptide was directly coated to the plate (y-axis) or when C-terminally biotinylated KQPA-peptide was captured via streptavidin coating of the plate (x-axis). B. Affinity of VH3-15/VL3-10 mAbs (n=13) to the indicated peptides representing the PfCSP N-terminal junction (KQPA and NPDP peptides) and central repeat region (NANP3 peptide) as measured by Surface Plasmon Resonance. Amino acid sequences of the respective peptides are indicated below the graph.

FIG. 4. Low sporozoite binding and inhibition observed for two representative high affinity VH3-15VL3-10 mAbs in contrast to high affinity NANP-reactive mAbs (2A10 and 317) with known parasite inhibitory activity. A. Histograms illustrate the binding intensity of VH3-33/VK1-5 (left) and VH3-15/VL3-10 (right) mAbs to Pb-PfCSP-transgenic sporozoites in flow cytometry. B. Percentage of sporozoites bound by selected VH3-15/VL3-10 (n=7) and VH3-33/VK1-5 (n=4) mAbs. C. Percent inhibition of the in vitro hepatocyte traversal activity of Pf sporozoites (spz) by the indicated mAbs at the indicated concentrations. mAbs 2A10 and mGO53 were used as positive and negative controls, respectively. Red lines indicate means. D. Ig gene features and KQPA peptide affinity of the two representative VH3-15/VL3-10 mAbs that were tested. Data represent at least two independent experiments. B. Mann-Whitney test. ** P<0.001.

FIG. 5. Fine epitope specificity of VH3-15/VL3-10 mAbs. A. Affinity of VH3-15 mAbs (n=13) to the indicated peptides representing the PfCSP N-junction (KQPA and NPDP) and central repeat region (NANP3) as determined by Surface Plasmon Resonance. B. Affinity of VH3-15/VL3-10 (circles) and VH3-33/VK1-5 (triangles) mAbs to the indicated peptides. Amino acid sequences of the peptides are illustrated.

FIG. 6. Crystal structure of 509893_121 Fab in complex with the KQPA peptide. 509893 Fab shown in surface representation with HC and LC framework regions in dark grey and white, respectively; and HCDR1, -2 and -3, and LCDR1, -2 and -3 highlighted in light blue, sky blue, density blue, pale green, lime, and forest, respectively. Bound KQPA peptide is shown as magenta sticks with residues labelled. Total buried surface area (BSA) of each peptide residue is listed in the table on the right. The contribution (40%) to peptide interactions by KQP residues is shown in red.

FIG. 7. Removal of KQP residues from 155 immunogen results in loss of binding by low-inhibitory VH3-15/VL3-10 mAbs. BLI binding curves of 155 and 203 nanoparticles to anti-KQPA specific Fabs 509893_121 and 511205_132. All samples were diluted in kinetics buffer to 10 μg/mL.

FIG. 8. Immunogens optimized at the N terminus with removal of KQP motif express well, are pure and show favorable biophysical characteristics. SDS-PAGE and negative stain electron micrographs (scale bar, 50 nm). BLI binding curves of 203 immunogens to anti-PfCSP Fabs 4493, 317, 1210, 1710, 5D5 and 509893_121. 1710 and 5D5 act as negative controls, since their epitopes are not present in this immunogen, whereas 4493, 317, and 1210 act as positive controls, since their epitopes are present in this immunogen. All samples were diluted in kinetics buffer to 10 μg/mL.

FIG. 9. Reactivity of sera derived from Kymice immunized with two different classes of immunogens (KQP-containing 155-A; KQP-lacking 203) at the indicated doses for a peptide containing the KQP residues. Serum IgG reactivity to a truncated N-terminal peptide (N-CSPshort) with the indicated amino acid sequence containing the KQP residues. Black lines indicate arithmetic mean. Data represent at least two independent experiments.

FIG. 10. Immunogenicity of immunogens with (155-A and 155-B) and without (203) KQP residues. Kymice (n=6 mice per group) were immunized with the indicated immunogens and doses in adjuvant or with adjuvant alone. Serum IgG responses against peptides with the indicated amino acid sequences representing the N-terminal junction and NANP repeat domain (P126; top) or NANP repeat domain (NANP, bottom) were measured by ELISA at different time points after the first (d0) or second immunization (d29). Mean and SD of the response over time are shown for each group (left) and serum IgG levels on d36 (right) for each mouse (circles). Lines in graphs on right indicate means. Data represent at least two independent experiments.

FIG. 11. Strong protection against challenge in wild-type mice after immunization with 203 immunogen that lacks KQP residues. Groups of wild type mice (n=5) were immunized intramuscularly with the 203 immunogen in adjuvant (red) following a prime-boost schedule (d0, d28) and three weeks later were exposed to three bites of Pb-PfCSP-infected mosquitoes. The adjuvant-only control group is shown in black. Parasitaemia was examined daily by flow cytometry of the blood samples.

FIG. 12. Immunogenicity of immunogens without KQP residues. WT mice (n=5 per group) were immunized i.m. with the indicated high-mannose glycosylated (203F+Kif) or non-glycosylated (211F) immunogens and doses (0.5 μg or 10 μg/mouse) in adjuvant or with adjuvant alone. Serum IgG responses against the indicated peptides were measured by ELISA at different time points after the first (d0) or second (d28) immunization. Data show the means of means with SD from three independent experiments with 5 mice per group.

FIG. 13: Immunogenicity of immunogens without KQP residues. WT mice (n=5 per group) were immunized i.m. with the indicated high-mannose glycosylated (203F+Kif) or non-glycosylated (211F) immunogens and doses (0.5 μg or 10 μg/mouse) in adjuvant or with adjuvant alone. Serum IgG responses against the indicated peptides were measured by ELISA at different time points after the first (d0) or second (d28) immunization. Data show the means with SD for 5 mice per group for three independent experiments.

FIG. 14: Highly protective serum responses induced by immunogens after removal of KQP residues. Sera inhibition activity induced by immunization (i.m.) with glycosylated (203F, green colors) or non-glycosylated (211F, blue colors) in adjuvant plotted against serum IgG titers against NANP₅ s. (A) Results of sporozoite traversal inhibition assays (N=3) at five pooled sera dilutions (days 35 (D35) and 51(D51)). Shown mean of three replicates +/−SEM. (B) Results of sporozoite traversal inhibition assays (N=3) at three sera dilutions (D35 and D51). Shown mean of the means per experiment +/−SEM. (C) In vivo protection data. Immunized mice and controls were exposed to bites of 3 infectious mosquitoes on day 52 after immunization. Blood parasitemia was tested for 10 days after infection. Pooled results of two independent experiments are shown (n=5 per group/per experiment).

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, the immunogens described herein, in aspects, exclude the C-terminal domain of PfCSP. In other aspects, the immunogens described herein specifically exclude a KQ or KQP motif. In aspects the immunogens described herein exclude an N-terminal KQ or KQP motif, or a KQ or KQP motif that is part of the N-terminal junction region in PfCSP.

In addition, all ranges given herein include the end of the ranges and also any intermediate range points, whether explicitly stated or not. With respect to the motifs described herein, which contain four amino acids, it will be understood that ranges of, for example, 0-20 repeats of these motifs encompass quarter integers as well, for example, 0.25, 0.5, and 0.75, and all other integer quarters, are contemplated herein. This indicates that the repeat is not necessarily complete and, for example, NANP may be repeated as NANPN, NANPNA, NANPNAN, NANPNANP, N, NA, NAN, NANP, A, AN, ANP, ANPN, N, NP, NPN, NPNA, P, PN, PNA, or PNAN, and so on.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The terms “protein nanoparticle” and “nanocage” are used interchangeably herein and refer to a multi-subunit, protein-based polyhedron shaped structure. The subunits or nanocage monomers are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, apoferritin, encapsulin, SOR, lumazine synthase, and pyruvate dehydrogenase are monomeric proteins that self-assemble into a globular protein complex that in some cases consists of 24, 60, 24, 60, and 60 protein subunits, respectively. Ferritin and apoferritin are generally referred to interchangeably herein and are understood to both be suitable for use in the fusion proteins, nanocages, and methods described herein. Carboxysome, vault proteins, GroEL, heat shock protein, E2P and MS2 coat protein also produce nanocages are contemplated for use herein. In addition, fully or partially synthetic self-assembling monomers are also contemplated for use herein.

It will be understood that each nanocage monomer may be divided into two or more subunits that will self-assemble into a functional nanocage monomer. For example, ferritin or apoferritin may be divided into an N- and C-subunit, divided substantially in half, so that each subunit may be separately bound to a different bioactive moiety for subsequent self-assembly into a nanocage monomer and then a nanocage. By “functional nanocage monomer” it is intended that the nanocage monomer is capable of self-assembly with other such monomers into a nanocage as described herein.

A “vaccine” is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine induces an immune response that reduces and/or prevents malaria disease compared to a control. In another non-limiting example, a vaccine induces an immune response that reduces the severity of the symptoms associated with malaria disease and/or decreases the parasite load compared to a control.

The term “antibody”, also referred to in the art as “immunoglobulin” (Ig), used herein refers to a protein constructed from paired heavy and light polypeptide chains, various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” and “immunogenic peptide” are used interchangeably herein and as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, such as mRNA, which comprises a nucleotide sequence or a partial nucleotide sequence encoding a protein that elicits an immune response therefore encodes an “antigen” as that term is used herein. Furthermore, one skilled in the art will understand that an antigen needs not be encoded solely by a full-length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen needs not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand and the non-coding strand, used as the template for transcription of a gene or cDNA, can be referred to as encoding the protein or other product of that gene or cDNA.

The skilled person will understand that the immunogens described herein can be provided as proteins or as viral vector vaccines or as mRNA vaccines, or any other vaccine format known. For example, COVID-19 mRNA vaccines are described in Gaviria, M., Kilic, B. A network analysis of COVID-19 mRNA vaccine patents. Nat Biotechnol 39, 546-548 (2021). https://doi.org/10.1038/s41587-021-00912-9, which is incorporated herein by reference, and the skilled person would appreciate that the immunogens described herein could be formulated in a similar way for administration.

The term “expression” as used herein is defined as the transcription and/or translation of a particular nucleotide sequence driven by a promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species, for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimens may be adjusted to provide the optimum therapeutic response, as is understood by a skilled person. For example, administration of an effective amount of the immunogens described herein is, in aspects, sufficient to increase immunity against a pathogen, such as Plasmodium.

Moreover, an immunization regime of a subject with an effective amount may consist of a single administration, or alternatively comprise a series of applications. The length of the immunization period depends on a variety of factors, such as the immunogen, the age of the subject, the dose of the agent, the responsiveness of the patient to the agent, or a combination thereof. It will also be appreciated that the effective dosage of the agent used for the treatment may increase or decrease over the course of a particular immunization regime. Changes in dosage may result and become apparent by standard assays known in the art. The immunogens described herein may, in aspects, be administered before, during or after treatment with conventional therapies for the disease or disorder in question, such as malaria.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

The term “adjuvant” refers to a compound or mixture that is present in a vaccine and enhances the immune response to an antigen present in the vaccine. For example, an adjuvant may enhance the immune response to a polypeptide present in a vaccine as contemplated herein, or to an immunogenic fragment or variant thereof as contemplated herein. An adjuvant can serve as a tissue depot that slowly releases the antigen and also as an immune activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS01/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL 1005), aluminum phosphates (e.g., AIPO.sub.4), R-848 (a Th1-like adjuvant), imiquimod, PAM3CYS, poly (I:C), loxoribine, BCG (bacille Calmette-Guerin) and Corynebacterium parvum, CpG oligodeoxynucleotides (ODN), cholera toxin derived antigens (e.g., CTA 1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

“Variants” are biologically active immunogens, fusion proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the immunogens described herein, wherein “biological” activity refers to a biological function (either inhibitory or stimulatory) caused by the immunogens.

The immunogens described herein may include modifications. Such modifications include, but are not limited to, conjugation to an effector molecule such as an anti-malaria agent or an adjuvant. Modifications further include, but are not limited to conjugation to detectable reporter moieties. Modifications that extend half-life (e.g., pegylation) are also included. Proteins and non-protein agents may be conjugated to the immunogens by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

A “Pan DR binding” peptide or “PADRE®” peptide (Epimmune, San Diego, Calif.) is a member of a family of molecules that binds more than one HLA class II DR molecule. The pattern that defines the PADRE® family of molecules can be referred to as an HLA Class II supertope. A PADRE® molecule binds to HLA-DR molecules and stimulates in vitro and in vivo human helper T lymphocyte (HTL) responses and can be referred to as providing exogenous T cell help. For a further definition of the PADRE® family, see for example, U.S. Ser. No. 09/709,774; Ser. No. 09/707,738; PCT publication Nos WO 95/07707, and WO 97/26784; U.S. Pat. Nos. 5,736,142; 5,679,640; and 6,413,935, each of which is incorporated herein by reference in its entirety.

### Immunogens

Described herein are malarial immunogens. Typically, the malarial immunogens comprise at least a portion of the wild-type PfCSP amino acid sequence but lack a KQ motif, more specifically a KQ motif that is found at or near the N-terminal junction region of the wild-type sequence. In other aspects, the malarial immunogen lacks a KQP motif and, more specifically, a KQP motif that is found at or near the N-terminal junction region of the wild-type sequence.

It has been surprisingly found that sequences lacking the KQ or KQP motifs resulted in an antibody response that was more focused to the junction/repeat region in the context of the human immunoglobulin repertoire. In contrast, PfCSP-based immunogens that contained the KQ or KQP motifs elicited a strong B cell response that is non-sporozoite-reactive and non-inhibitory. The Examples herein demonstrate that this unexpected and highly desirable improved immunogenic effect is clearly tied to the lack of the KQ or KQP motifs.

In additional aspects, the malarial immunogen may lack an N-terminal ETG sequence.

For example, the malarial immunogens in aspects comprise at least one of the following motifs: NPDP, NANP, NVDP, and NANP, which can be present in any order and repeated in order or not to any extent. As described above, the malarial immunogens lack a KQ or KQP motif, particularly one that may be found upstream of any of the NPDP, NANP, NVDP, and/or NANP motifs. The malarial immunogens described herein are immunogenic and find use in the treatment and/or prevention of malaria. In particular aspects, the malarial immunogens find use in vaccines for preventing malaria.

In certain aspects, the malarial immunogens comprise one or more repeat motifs derived from PfCSP, such as:
NPDPₐ; PDPNₐ; DPNPₐ; or PNPDₐ;
NANP_{b}; ANPN_{b}; NPNA_{b}; or PNAN_{b};
NVDP_{c}; VDPN_{c}; DPNV_{c}; or PNVD_{c}; and
NANP_{d}; ANPN_{d}; NPNA_{d}; or PNAN_{d}.

The letters a, b, c, and d designate how many times the given motif is repeated and each of a, b, c, and d are independently present or absent and, if present, can be repeated any desired number of times as long as the resultant malarial immunogen remains immunogenic. Typically, at least two motifs are present, such that a+b+c+d is at least 2.

Typically a, b, c, d, or any combination thereof are each independently at least about 1 and more typically, a, b, c, d, or any combination thereof are each independently from about 1 to about 40. This means that a, b, c, and d are each usually present and are each typically either not repeated or repeated up to about 40 times. Fractional repeats are understood to be included herein, as each motif comprising 4 amino acid, therefore a 1.25 repeat would be understood to include the original motif with the first amino acid repeated, for example, NANPN. Likewise, a 1.5 repeat would represent for example NANPNA, and a 1.75 repeat would represent for example NANPNAN.

From the above, it will be understood that a, b, c, and d are each independently present or absent and optionally repeated any number of times. Typically, however, each of a, b, c, and d are each present and are independently from about 1 to about 100, such as from about 1, about 2, about 3, about 4, about 5, about 6, about 7, about 8, about 9, about 10, about 11, about 12, about 13, about 14, about 15, about 16, about 17, about 18, about 19, or about 20 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 60, about 70, about 75, about 80, about 90, or about 100, such as from about 1 to about 40, or about 1 to about 20, or from 1 to about 10. Typically, a is 1, b is 1, c is 3, d is 5 or 18.5.

It will be understood that when any given motif is repeated in whole or in part, i.e., when a, b, c, d, or e is greater than 1, the repeated motif may be contiguous or non-contiguous with the original motif. For example, for NANP2, this may be NANPNANP or NANP-intervening sequence-NANP. There may be combinations of contiguous or non-contiguous repeated motifs as well, such as, for example, NANPNANP-intervening sequence-NANP.

It will be understood that the motifs listed above and their respect repeats if present may be in any order, however, the motifs are typically in the order NPDPₐ-NANP_{b}-NVDP_{c}-NANP_{d}.

In certain aspects, the motifs described herein may be preceded by other motifs, such as, for example, an ADG or PADG motif. These motifs if included are typically at or near the N-terminus. In some aspects, the ADG or PADG motif may repeat together with one or more of the NPDPₐ, NANP_{b}, NVDP_{c}, or NANP_{d} motifs referred to above. For example, ADGNPDP may be a repeating unit, PADGNPDP may be a repeating unit, and any of the other permutations of motifs described herein may be combined with ADG or PADG similarly.

The variant sequences or fragments described herein may have any desired sequence identity to the comparator sequences herein, as long as they retain at least some level of the desired function of the comparator sequence. For example, the malarial immunogens described herein are immunogenic and variants of these peptides would retain at least some immunogenicity. Typically, variants comprise at least about 70%, 75%, 80%, 85%, 90%, 95%, 97%, or 99% sequence identity to the immunogenic peptide or to the wild-type PfCSP amino acid sequence lacking the KQ motif.

In certain aspects, the malarial immunogens described herein are fused directly or indirectly to a nanocage monomer peptide. The nanocage monomers described herein can be any of the nanocage monomers as described in, for example, WO/2019/023811, which is incorporated herein by reference. Typically, the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof and may be provided as two or more self-assembling subunits.

The nanocage monomer may be derived from any species source, but typically is from Helicobacter pylori and is typically ferritin, termed HpFerr for short. Also typically, the nanocage monomer peptide is not human. In this way, anti-self immune responses can be mitigated.

Typically, the nanocage monomer peptide comprises or consists of the amino acid sequence:

MLSKDIIKLLNEQVNKEMNSSNLYMSMSSWCYTHSLDGAGLFLFDHAAEE
YEHAKKLIVFLNENNVPVQLTSISAPEHKFEGLTQIFQKAYEHEQHISES
INNIVDHAIKSKDHATFNFLQWYVAEQHEEEVLFKDILDKIELIGNENHG
LYLADQYVKGIAKSRKS.

Similar to above, functional variants or fragments are also included. Typically, variants have at least 70% sequence identity to the reference sequence and variants and fragments are capable of self-assembly into a nanocage.

It will be understood that the nanocage monomer peptide may be modified in a variety of different ways in order to reduce an anti-nanocage monomer peptide immune response and augment the response to the desired antigenic component. For example, the nanocage monomer peptide may at least partially or fully covered, for example, partially or fully glycan covered to mask B cell epitopes of the nanocage. Thus, the nanocage monomer may comprise at least one NXT and/or NXS glycosylation motif. For example, the sequence noted above (or a variant or fragment thereof) may be modified to comprise one or more of a K77N, a E79T, a E99N, and an I101T mutation. For example, the nanocage monomer may be glycosylated at N79 and/or N99, such as N79 and N99.

Thus, in aspects, it will be understood that the nanocage monomer is glycosylated. It can be glycosylated with N-linked glycans, O-linked glycans, combinations of different glycans, and/or glycans from production in other various expression systems, such as yeast, insect cells, plants, etc. In typical aspects, the nanocage monomer is glycosylated with high-mannose glycans.

Typically, the nanocage monomer peptide is selected so that a plurality of the nanocage monomer peptides self-assemble into a nanocage. It will be understood that the immunogenic peptide may decorate the interior and/or exterior surface of the nanocage.

The malarial immunogens described herein may comprise additional peptide sequences. For example, a peptide providing exogenous T cell help and/or a peptide that provides autologous T cell help may be fused to the other peptides described herein in any order. In aspects, the peptide that provides exogenous T cell help comprises a PADRE peptide and/or a peptide derived from a pathogenic molecule, such as a tetanus toxoid peptide. If a PADRE peptide is used, it typically comprises the amino acid sequence AKFVAAWTLKAAA, or a functional variant thereof having at least 70% sequence identity thereto or a fragment of either thereof. In alternate or additional aspects, a peptide providing autologous T cell help may be included herein. Typically, the peptide that provides autologous T cell help comprises a PfCSP T cell peptide epitope.

As described above with respect to the immunogenic peptide, the peptide that provides exogenous T cell help and/or the peptide that provides autologous T cell help may independently decorate the interior and/or exterior surface of the assembled nanocage, and this may be the same or different from the way in which the immunogenic peptide decorates the nanocage.

In certain aspects, the malarial immunogens described herein comprise one or more flexible or inflexible linkers between one or more of the motifs, the nanocage monomer, and any further peptides, such as the peptide that provides exogenous T cell help and/or the peptide that provide autologous T cell help. Typically, the linker is sufficiently flexible to allow the immunogenic peptide to adopt a favourable conformation, once the protein is expressed.

The linker is generally long enough to impart some flexibility to the antigen, although it will be understood that linker length will vary depending upon the antigen and antibody sequences and the three-dimensional conformation of the malarial immunogens. Thus, the linker is typically from about 1 to about 30 amino acid residues, such as from about 1, 2, 3, 4, 5, 6, 7, 8, 9, 10, 11, 12, 13, 14, 15, 16, 17, 18, 19, 20, 21, 22, 23, 24, 25, 26, 27, 28, or 29 to about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, such as from about 8 to about 12 amino acid residues, such as 8, 10, or 12 amino acid residues.

The linker may be of any amino acid sequence that does not interfere with the binding of the immunogenicity of the immunogenic peptide. In one typical example, the flexible linker comprises GGS or a GGS repeat, for example, GGSGGSGGSG, GGGGSGGSGGSGGS, or GGGGGSGGSGGSGGS.

Specific examples of malarial immunogens described herein include, for example, fusion proteins comprising or consisting of the sequence:

ADG-NPDP-NANPNVDP3-NANP5-Hpferr-PADRE;
ADG-NPDP-NANPNVDP3-NANP18-Hpferr-PADRE;
ADG-NPDP-NANPNVDP3-NANP18.5-Hpferr-PADRE;
ADG-NPDP-NANPNVDP3-NANP5-LS-PADRE;
ADG-NPDP-NANPNVDP3-NANP18-LS-PADRE;
and/or
ADG-NPDP-NANPNVDP3-NANP18.5-LS-PADRE.

More specifically, certain examples include fusion proteins comprising or consisting of the amino acid sequence:

203:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHNFTGLTQIF
QKAYEHEQHISNSTNNIVDHAIKSKDHATFNFLQWYVAEQHEEEVLFKDI
LDKIELIGNENHGLYLADQYVKGIAKSRKSGGSASAKFVAAWTLKAAA
205:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHNFTGLTQ
IFQKAYEHEQHISNSTNNIVDHAIKSKDHATFNFLQWYVAEQHEEEVLFK
DILDKIELIGNENHGLYLADQYVKGIAKSRKSGGSASAKFVAAWTLKAAA
206:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFK
DILDKIELIGNENHGLYLADQYVKGIAKSRKSGGSASAKFVAAWTLKAAA
207:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHNFTGLTQIF
QKAYEHEQHISNSTNNIVDHAIKSKDHATFNFLQWYVAEQHEEEVLFKDI
LDKIELIGNENHGLYLADQYVKGIAKSRKS
208:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHNFTGLTQ
IFQKAYEHEQHISNSTNNIVDHAIKSKDHATFNFLQWYVAEQHEEEVLFK
DILDKIELIGNENHGLYLADQYVKGIAKSRKS
209
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDI
LDKIELIGNENHGLYLADQYVKGIAKSRKS
210:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFK
DILDKIELIGNENHGLYLADQYVKGIAKSRKS
211:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDI
LDKIELIGNENHGLYLADQYVKGIAKSRKSGGSASAKFVAAWTLKAAA
212:
ADGNPDPNANPNVDPNANPNVDPNANPNVDPNANPNANPNANPNANPNAN
PGGSGGSGGSGGSGGSDIIKLLNEQVNKEMQSSNLYMSMSSWCYTHSLDG
AGLFLFDHAAEEYEHAKKLIIFLNENNVPVQLTSISAPEHKFEGLTQIFQ
KAYEHEQHISESINNIVDHAIKSKDHATFNFLQWYVAEQHEEEVLFKDIL
DKIELIGNENHGLYLADQYVKGIAKSRKSGGSASAKFVAAWTLKAAA
213:
ADGNPDPNANPNVDPNANPNVDPNANPNVDPNANPNANPNANPNANPNAN
PGGSGGSGGSGGSGGSDIIKLLNEQVNKEMQSSNLYMSMSSWCYTHSLDG
AGLFLFDHAAEEYEHAKKLIIFLNENNVPVQLTSISAPEHNFTGLTQIFQ
KAYEHEQHISNSTNNIVDHAIKSKDHATFNFLQWYVAEQHEEEVLFKDIL
DKIELIGNENHGLYLADQYVKGIAKSRKSGGSASAKFVAAWTLKAAA

or a functional variant having at least 70% sequence identity thereto, or a functional fragment of either thereof.

Also described herein are nucleic acid molecules encoding the malarial immunogens described herein, vectors, host cells, and vaccines comprising the malarial immunogens described herein. As will be understood, vaccines may include, for example, adjuvants, as further described above.

The malarial immunogens described herein are immunogenic and are capable of eliciting an immune response in a subject. Thus, in aspects, antibodies that bind to the malarial immunogens described herein are also contemplated. Methods of immunizing subjects, including humans and animals, in order to produce and characterize such antibodies are known. Such antibodies can then be used in assays, therapeutic or preventative compositions, etc.

The malarial immunogens described herein are, in aspects, useful as a highly efficacious pre-erythrocytic subunit malaria vaccine. These are also or alternatively, in aspects, a C-terminal truncated PfCSP antigen. In addition or alternately, the fusion proteins described herein are useful in providing a boostable malaria vaccine that is PfCSP-based.

Further described herein is a glycan-covered nanocage monomer peptide, which may be as described above. For example, the nanocage monomer is typically ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof. The nanocage monomer may be provided as two or more self-assembling subunits. Typically, the nanocage monomer peptide is from Helicobacter pylori and/or is not human.

It will be understood that the nanocage monomer peptide may further comprise a bioactive moiety, such as an antibody or fragment thereof, an antigen, a detectable moiety, a pharmaceutical agent, a diagnostic agent, or combinations thereof. Typically, the bioactive moiety comprises an antigen.

A plurality of the nanocage monomer peptides typically self-assemble into a nanocage and the bioactive moiety decorates the interior and/or exterior surface of the nanocage.

As described above, the nanocage monomer peptide is at least partially or fully covered or masked, typically glycan covered. Generally, the nanocage monomer comprises at least one NXT and/or NXS glycosylation motif and/or comprises the amino acid sequence:

MLSKDIIKLLNEQVNKEMNSSNLYMSMSSWCYTHSLDGAGLFLFDHAAEE
YEHAKKLIVFLNENNVPVQLTSISAPEHKFEGLTQIFQKAYEHEQHISES
INNIVDHAIKSKDHATFNFLQWYVAEQHEEEVLFKDILDKIELIGNENHG
LYLADQYVKGIAKSRKS

or a functional variant having at least 70% sequence identity thereto, or a functional fragment of either thereof, and wherein the sequence comprises at least one of a K77N, a E79T, a E99N, and an I101T mutation.

It will be understood that the malarial immunogens may be modified as described above as a general concept and/or in the interest of immuno-modulation or immuno-focusing. Further, T-cell epitope linear peptides may be included that help immuno-modulate/increase humoral/antibody responses.

As described herein, a substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that match when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The malarial immunogens of the present invention may also comprise additional sequences to aid in their expression, detection or purification. Any such sequences or tags known to those of skill in the art may be used. For example, and without wishing to be limiting, the malarial immunogens may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

The malarial immunogens may also be in a multivalent display. Multimerization may be achieved by any suitable method of known in the art. For example, and without wishing to be limiting in any manner, multimerization may be achieved using self-assembly molecules as described in Zhang et al (2004a; 2004b) and WO2003/046560.

Also encompassed herein are isolated or purified malarial immunogens, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the malarial immunogens may be linked to a cargo molecule or the assembled nanocages may hold a cargo molecule; the fusion proteins may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the malarial immunogens may be linked to a fluorescent agent such as FITC or may genetically be fused to the Enhanced Green Fluorescent Protein (EGFP).

Antibodies against the malarial immunogens described herein specifically bind to the malarial immunogens. Antibody specificity, which refers to selective recognition of an antibody for a particular epitope of an antigen, for the malarial immunogens described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, or 10⁻⁹ M.

Also described herein are nucleic acid molecules encoding the malarial immunogens and polypeptides described herein, as well as vectors comprising the nucleic acid molecules and host cells comprising the vectors.

Polynucleotides encoding the malarial immunogens described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Additionally, the expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colE1, pCR1, pBR322, pMB9, pUC, pKSM, and RP4. Prokaryotic vectors also include derivatives of phage DNA such as MI3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA. Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The malarial immunogens described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce malarial immunogens by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The malarial immunogens described herein can be fused to additional amino acid residues. Such amino acid residues can be a peptide tag to facilitate isolation, for example. Other amino acid residues for homing of the malarial immunogens to specific organs or tissues are also contemplated.

In another aspect, described herein are methods of vaccinating subjects by administering an effective amount of the malarial immunogens described herein to a mammal in need thereof, typically an adult, a young, a juvenile, or a neonatal mammal. As described above, an effective amount means an amount effective to produce the desired effect, such as providing a protective immune response against the antigen in question that mediates protection from Plasmodium falciparum.

Any suitable method or route can be used to administer the malarial immunogens and vaccines described herein. Routes of administration include, for example, oral, intravenous, intraperitoneal, subcutaneous, or intramuscular administration.

It is understood that the malarial immunogens described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be generated using the malarial immunogens described herein for administration to other mammals as well. The term “mammal” as used herein is intended to include, but is not limited to, humans, laboratory animals, domestic pets and farm animals.

Also included herein are kits for vaccination, comprising a therapeutically or prophylactically effective amount of a malarial immunogen described herein. The kits can further contain any suitable adjuvant for example. Kits may include instructions.

The above disclosure generally describes the present invention. A more complete understanding can be obtained by reference to the following specific examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1

### Summary

PfCSP is a vaccine target against malaria that seeks to elicit anti-infective immunity. We designed PfCSP-based immunogens in which the KQP amino acids in the N-terminal junction of the natural PfCSP sequence were removed and the N terminus of the immunogen was optimized. Such immunogens induced strong and protective immune responses in WT mice, and interestingly much stronger immune responses in the context of a human immunoglobulin repertoire compared to an immunogen that contains these KQP amino acids. These data suggest that a PfCSP immunogen lacking these Lys and/or Gln and/or Pro amino acids may elicit stronger and more protective immunity against malaria in humans upon vaccination.

### Background

Malaria is a major global health concern, with over 400,000 deaths and 228 million cases annually, a majority of which are attributed to Plasmodium falciparum (Pf) (1). In recent years, progress in combating the disease has halted, predominantly due to the increase in resistance of mosquito vectors to insecticides (2) and the emergence of multidrug-resistant parasites (3).

The circumsporozoite protein (PfCSP) densely covers the surface of Plasmodium sporozoites. It plays a critical role in parasite development in the Anopheles mosquito vector and establishment of infection in human liver cells (4-7). PfCSP contains a largely disordered central region composed of only five amino acids (aa; asparagine (Asn, N), alanine (Ala, A), valine (Val, V), aspartate (Asp, D) and proline (Pro, P)) arranged in a large number of repeating NANP and NANP-like motifs (NPDP and NVDP) (8,9). The central region is flanked by an N-terminal domain, containing a conserved five-amino-acid motif named Region I (KLKQP in positions 93-97) followed by residues ADG, and a C-terminal thrombospondin repeat (TSR) domain that anchors the protein to the sporozoite surface via a glycosylphosphatidylinositole (GPI) anchor. Unlike N- and C-terminal domains, which harbor substantial sequence diversity, the central region of repeating sequence displays slight variability only in its number of NANP and NVDP motifs (10,11).

Exposed on sporozoites, NANP repeats are immunodominant and antibodies against the central repeats can protect from infection in humans (12). Infection with Pf sporozoites appears to drive anti-NANP antibody responses in humans through clonal selection of B cells with high affinity germline B-cell receptors (BCRs) dominated by cells expressing VH3-33/VK1-5 genes (13). In addition to antibodies against NANP repeats, some studies also identified potent antibodies that preferentially target the junction region that links the PfCSP N-terminus and the NANP repeats region, which contain a single NPDP motif and several interspersed NVDP motifs (14,15). Cross-reactivity across repeat motifs is a feature of antibodies encoded by different Ig-gene combinations, and it was also observed for murine mAbs targeting CSP sequences from other species of Plasmodium, including P. berghei (16) and P. vivax (17). Moreover, cross-reactivity appears to be associated with high binding affinity and parasite inhibition activity, and thus repeated antigen exposure has been shown to result in the enrichment of cross-reactive antibodies (18,19). Antibody selection in humans from sporozoite exposure is likely driven by affinity to NANP, rather than to NVDP or NPDP motifs (18,19), due to the higher number of NANP repeats in PfCSP and differences in immunogenicity and accessibility of PfCSP epitopes (20,21).

An open question is how to design a PfCSP-based immunogen that will elicit the most potently inhibitory antibody response to protect against malaria parasite infection and disease. Indeed, a broadly effective malaria vaccine against Pf has remained elusive with only a single vaccine, RTS,S/AS01, having recently been approved for use after pilot implementation studies in Malawi, Ghana, and Kenya. RTS,S/AS01, and the similar R21/Matrix M vaccine, are based on many PfCSP NANP repeats and the C-terminal domain fused to the human hepatitis B surface antigen (HBsAg), without inclusion of the PfCSP N-terminal or junction sequences. Although these vaccines elicited protection from severe disease shortly after multiple vaccinations, this immunity waned rapidly (22,23), highlighting a need for improved PfCSP immunogens. Antibody research as described above informs that in addition to the NANP repeats contained in such immunogens as RTS,S and R21, inclusion of the upstream junctional epitopes (NPDP and NVDP motifs) could bring antibodies into the response that are more inhibitory. However, it remains unclear which region upstream of the NPDP/NVDP/NANP junction/repeats should be included in a next-generation PfCSP immunogen.

Unexpectedly, we found in the current work that PfCSP-based immunogens that contain amino acids “KQP”—which are part of the highly conserved Region I immediately upstream of the central region—elicited upon immunization against a human immunoglobulin repertoire a strong antibody response that is non-sporozoite-reactive and non-inhibitory. Guided by newly-obtained structure-function relationships of atomic precision, we engineered an optimized PfCSP-based immunogen that removes this undesired epitope. We demonstrate that elimination of the KQP motif in PfCSP immunogens helps focus the antibody response to the junction/repeat region in the context of the human immunoglobulin repertoire and in non-human repertoires. The cells dominate the response, especially in germinal centers, which are the place where high affinity responses to vaccines are generated.

Thus, the PfCSP immunogens described here are a careful combination of not only regions associated with recognition by the most potent antibodies (junction/repeat), but also remove undesirable epitopes associated with recognition by non-sporozoite reactive, non-inhibitory antibodies (e.g. N-term, C-term, KQP) that can distract the immune response. Such immunogens have the potential to elicit the strongest inhibitory immune response in humans against malaria parasite infection.

### Materials and Methods

### Expression and Purification of Immunogens

Genes encoding immunogens were synthesized by GeneArt (Life Technologies) and cloned into the pHLsec or pcDNA3.4 expression vectors. Secreted immunogens were expressed in CHO-M or HEK 293 cells (ThermoFisher Scientific) from stable cell pools or from transient transfection using 50 μg of filtered DNA and the transfection reagent FectoPRO (Polyplus Transfections) at a 1:1 ratio. Harvested supernatant was filtered through a LV Centramate™ Lab Tangential Flow Filtration System of 100 MWCO (Pall Laboratory) while buffer exchanging from Expression Medium to 20 mM sodium acetate pH 4 buffer. The resulting protein solution was loaded into a HiTrap Q HP column (anion exchange) (Cytiva) and eluted with 1 M NaCl. Fractions containing the protein were desalted into 1×PBS with HiTrap Desalting columns (Cytiva). Ammonium sulfate was added to the solution to a final concentration of 1.5 M for further purification with a HiTrap Phenyl HP column (Hydrophobic purification) (Cytiva) equilibrated with 1×PBS, 1.5 M ammonium sulfate buffer. The protein was eluted in a gradient with 1×PBS buffer. Fractions containing the protein were again desalted into 1×PBS with HiTrap Desalting columns (Cytiva). Purified protein was concentrated to 2 mg/ml and filtered with a 0.22 μm membrane for sterility. SDS-PAGE was run with 1 μg of protein in reducing and acidic (glycine pH 2.2) conditions.

### Fab Production and Purification

VH and VL regions of mAbs 4493 (Murugan et al., 2020), 317 (Oyen et al., 2017), 1210 (Imkeller et al., 2018), and 5D5 (Thai et al., 2020) were individually cloned into pcDNA3.4-TOPO expression vectors immediately upstream of human Igγ1-CH1 and Igκ domains, respectively. VH and VL regions of mAb 509893 were individually cloned into pcDNA3.4-TOPO expression vectors immediately upstream of human Igγ1-CH1 and IgA domains, respectively. Paired Fab heavy and light chain plasmids were co-transfected into HEK293F cells (Thermo Fisher Scientific) for transient expression and purified via KappaSelect (4493, 317, 1210, 5D5) or LambdaFabSelect (509893) affinity chromatography (Cytiva) and cation exchange chromatography (MonoS; Cytiva). 4493, 317, 1210 and 5D5 Fabs were purified through an additional size-exclusion chromatography step (Superdex 200 Increase 10/300 GL; Cytiva).

### IgG Production and Purification

1710 IgG (Scally et al., 2018) was transiently expressed in HEK293F cells by co-transfection of paired Ig heavy and light chains, and purified through protein A affinity chromatography (Cytiva), followed by size-exclusion chromatography (Superdex 200 Increase 10/300 GL; Cytiva).

### Biolayer Interferometry (BLI)

BLI (Octet RED96, ForteBio) experiments were performed to evaluate the binding of immunogens to anti-CSP IgGs/Fabs 4493, 317, 1210, 1710, 5D5 and to anti-KQP Fabs 509893 and 511205. All samples were diluted in kinetics buffer (PBS, pH 7.4, 0.01% [w/v] BSA, 0.002% [v/v] Tween-20) to 10 μg/mL and Fabs were immobilized onto anti-human Fab-CH1 biosensors (ForteBio). After a stable baseline was established, biosensors were dipped into wells containing immunogens in kinetics buffer.

### Negative-Stain Electron Microscopy

Immunogens at a concentration of ˜0.03 μg/mL were deposited onto carbon film-coated grids and stained with 2% uranyl formate. Grids were imaged with a FEI Tecnai T20 electron microscope operating at 200 kV with an Orius charge-coupled device (CCD) camera (Gatan Inc).

### Mice and Immunizations

Studies used either Kymab mice transgenic for human immunoglobulin heavy, lamda and kappa gene segments (Lee et al. Nat Biotechnol 32, 356-363 (2014))., all females or males and females, typically ranging between 8-12 weeks old (all mice within 2 weeks of age) at the time of immunization (defined as the day dose 1 is administered) or female 7- to 9-week-old C57BL/6J mice purchased from commercial vendors. Immunogens were mixed 1:1 with Sigma Adjuvant System (SAS, Sigma) or TLR-4 agonist-based adjuvants according to manufacturer's instruction. Mice were immunized according to the immunization schedule and immunogen dose mentioned in the figure legends. Live single B cells identified in flow cytometry as germinal center B cells (CD95+GL7+) or plasma cells (TACI+CD138+) from lymph node and bone marrow of the immunized mice were sorted. For both populations, PfCSP positivity was assigned based on B cells binding to fluorochrome labelled recombinant PfCSP. Paired Ig genes sequences of the heavy and light chains were obtained by performing single cell RT-PCR and next generation sequencing of the indexed amplicons.

### Sequence Analysis:

Ig sequence assembly and annotation were performed using sciReptor, a bioinformatic pipeline for flow cytometry index data and sequence data integration (Imkeller et al, BMC Bioinformaticps://doi.org/10.1186/s12859-016-0920-1). Downstream sequence analyses and data visualization were performed using R 4.1.1.

### Monoclonal Antibody Production:

Selected Ig genes were synthesized, cloned into respective IGH, IGK or IGL expression vectors and monoclonal antibodies were recombinantly expressed using HEK293 cells.

### Crystallization and Structure Determination

Purified 509893 Fab was concentrated and diluted to 10 mg/mL with KQPA peptide in a 1:3 molar ratio. The 509893 Fab/KQPA complex was then mixed in a 1:1 ratio with 0.2 M lithium sulfate, 25% (w/vol) PEG 3350, 0.1 M bis-Tris, pH 5.5. Crystals appeared after ˜3 days and were flash-frozen in liquid nitrogen. Data were collected at the 23-IDB beamline at the Advanced Photon Source, processed, and scaled using XDS (Kabsch, 2010). The structure was determined to 2.40 Å resolution by molecular replacement using Phaser (McCoy et al., 2007). Refinement of the structure was performed using phenix.refine (Adams et al., 2010) and iterations of refinement using Coot (Emsley et al., 2010). Buried surface area (BSA) was calculated using the protein interfaces, surfaces and assemblies (PISA) service at the European Bioinformatics Instp://www.ebi.ac.uk/pdbe/prot_int/pistart.html; Krissinel and Henrick, 2007).

### Antibody Affinity Measured Using SPR

SPR-based assays were performed to determine the affinity of antibodies using a Biacore T200 system and Biacore sensor chip CM5. Two flow cells were immobilized with anti-human IgG antibodies using human Fab capture kit by following manufacturer's instructions. Antibody samples (10 μg/ml) as well as the negative control mG053 (10 μg/ml) were captured in the sample and reference flow cells, respectively. Stabilization of both flow cells was performed by SPR running buffer at 10 μl/min flow rate for 10 min. A serial dilution of the indicated peptides was performed in SPR running buffer and the following concentrations were injected into both flow cells: 0 nM, 15.4 nM, 92.6 nM, 555.5 nM, 3333.3 nM and 20,000 nM using a flow rate of 30 μl/min. Dissociation and association took place at 25° C. for 60 s and 180 s, respectively. Between the injections of different sample antibodies, flow cells were regenerated using 10 mM glycine in HCl. Data was analyzed using a 1:1 binding model or steady-state kinetic analysis with Biacore T200 software V2.0.

### Plasmodium Parasites

Plasmodium falciparum NF54 (a kind gift of R. Sauerwein) were cultured in O+ human red blood cells at 37° C., 4% CO₂, 3% O₂ in Heracell 150i Tri-gas incubators (Thermo Scientific). For gametocyte production, asynchronous parasite cultures were diluted to 1% parasitaemia and maintained for 15-16 days with daily change of RPMI-1640 medium (Thermo Scientific) supplemented with 10% human A+ serum and 10 mM hypoxantine (c-c-Pro) until mosquito infections. Pb-PfCSP, a replacement P. berghei line expressing PfCSP (NF54) under the control of the Pb CSP regulatory sequences¹⁹, was obtained from Chris J. Janse and Shahid M. Khan and passaged every 3-4 days in CD1 female mice.

### Mosquitoes

All mosquitoes were kept at 28-30° C. and 70-80% humidity. Anopheles coluzzii Ngousso S₁ strain were used for the production of Pf NF54 sporozoites. A. gambiae 7b line, immunocompromised transgenic mosquitoes derived from the G3 laboratory strain, were used for production of Pb-PfCSP sporozoites and for in vivo mosquito challenge experiments.

### Pb-PfCSP Sporozoites FACS

Anopheles. gambiae 7b mosquitoes were fed on female CD1 mice infected with Pb-PfCSP parasites (0.1-0.8% gametocytemia) and kept at 20° C. and 80% humidity until further usage. Infected mosquitoes were offered an additional uninfected blood meal at 7 days post infection (dpi), and 20 mosquitoes were dissected for oocyst counts at 17 dpi. Pb-PfCSP sporozoites were isolated from mosquito salivary glands on 18 dpi. Siliconized microtubes (Alpha Laboratories) and pipet tips (VWR) were used to minimize sporozoites binding to the surface. Monoclonal antibodies at the indicated concentrations were incubated with 150,000 sporozoites in a total volume of 100 μl PBS with 1% FCS for 30 min at 4° C. Upon washing, the sporozoites were incubated with anti-human IgG1-Cy5 (DRFZ, Berlin) at 2 μg/ml in PBS with 1% BSA for 30 min at 4° C. After washing, the live sporozoites were identified by GFP expression and mAb binding was quantified using FACS LSR II instrument (BD Biosciences). Data analysis was performed using FlowJo V.10.0.8 (Tree Star).

### Pf Hepatocyte Traversal Assay

Anopheles coluzzii mosquitoes were infected with mature Pf gametocyte (NF54 strain) cultures via artificial midi-feeders (Glass Instruments, the Netherlands) and kept in a controlled S3 facility in accordance with the local safety authorizations (Landesamt für Gesundheit und Soziales Berlin, Germany, LAGeSo, project number 411/08). Pf sporozoites were collected 13-15 days post infection from the mosquito salivary glands in HC-04 medium and used in hepatocyte traversal assay as described previously (24). Briefly, salivary gland Pf sporozoites in HC-04 medium were pre-incubated with mAbs at the indicated concentration in 27.5 μL for 30 min on ice and added to human hepatocytes (HC-04; (25)) for 2 h at 37° C. and 5% CO₂ in the presence of 0.5 mg mL⁻¹ dextran-rhodamine (Molecular Probes). Cells were washed and fixed with 1% PFA in PBS before flow cytometry measurements of dextran positivity using FACS LSR II instrument (BD Biosciences). Data analysis was performed by subtraction of the background (dextran positivity in cells treated with uninfected mosquito salivary gland material) and normalization to the maximum Pf traversal capacity (dextran positivity in cells treated with salivary gland Pf sporozoites) using FlowJo V.10.0.8 (Tree Star). A chimeric humanized version of the PfCSP-reactive mAb 2A10 (24) and of the non-PfCSP-reactive mAb mG053 (26) were used as positive and negative controls, respectively.

### In Vivo Mosquito Challenge

A. gambiae 7b mosquitoes were fed on female CD1 mice infected with Pb-PfCSP parasites (0.1-0.8% gametocytemia) and kept at 20° C. and 80% humidity for one week and offered an additional uninfected blood meal. On day 18, mosquitoes with fluorescence in the area of the salivary glands were selected by fluorescence stereoscope and 3 females were placed into individual cups. On the next day, the immunogen-injected mice were anesthetized and placed on the cups until all females have taken a blood meal. The mice were bled starting from the day 3 post challenge and the blood samples were examined for the infected red blood cells by flow cytometry using FACS LSR II instrument (BD Biosciences). Mice injected with adjuvant alone were used as a negative control.

### Enzyme-Linked Immunosorbent Assay (ELISA)

ELISA was performed following standard procedures. In brief, ELISA plates were coated with antigen in PBS over night at 4° C. Plates were washed 3 times with 0.05% Tween in PBS before blocking with 4% BSA in PBS for 1 h at RT. After washing with 0.05% Tween in PBS, diluted serum samples in 1% BSA/PBS were added and incubated for 1.5 h at RT. After washing with 0.05% Tween in PBS, secondary goat-anti-mouse IgG or IgM HRP antibody in blocking buffer was added and incubated for 1 h at RT. After washing with 0.05% Tween in PBS, ABTS substrate solution was added. Absorbance was measured at 405 nm. Binding of recombinant mAbs to the antigens were performed following similar steps, serial dilution of mAbs at 0.01, 0.06, 0.25 and 1.0 μg/ml was used for binding to antigen and goat anti-hIgG-HRP was used as secondary antibody.

### Results and Discussion

Immunizations in WT mice and Kymice (transgenic for the human Ig antibody repertoire) were performed with different immunogens and administration schedules e.g. 1) PfCSP sequences fused to different nanoparticle (lumazine synthase or Helicobacter pylori (Hp) ferritin); 2) PfCSP-based immunogens containing or not PfCSP cross-reactive epitopes (NPDP, NVDP) and containing or not efficient T-cell help (e.g. PADRE epitope), and of varying NANP repeat lengths; 3) immunogens 20 containing or not two non-native N-linked glycosylation sites on the Hpferritin protomer (N79 and N99) in attempts to decrease anti-carrier antibody responses and increase anti-PfCSP titers; and 4) homologous or heterologous prime-boosts with different schedules, administration routes, regimens and adjuvants. Analyses of immune cell frequencies, sequencing data and expressions of selected mAbs recovered from these experiments were performed. Sera and individual mAbs were evaluated for avidity/affinity measurements to different PfCSP sequences, in traversal inhibition assays, and for in vivo liver burden measurements and parasitemia challenge using PfCSP transgenic P. berghei parasites.

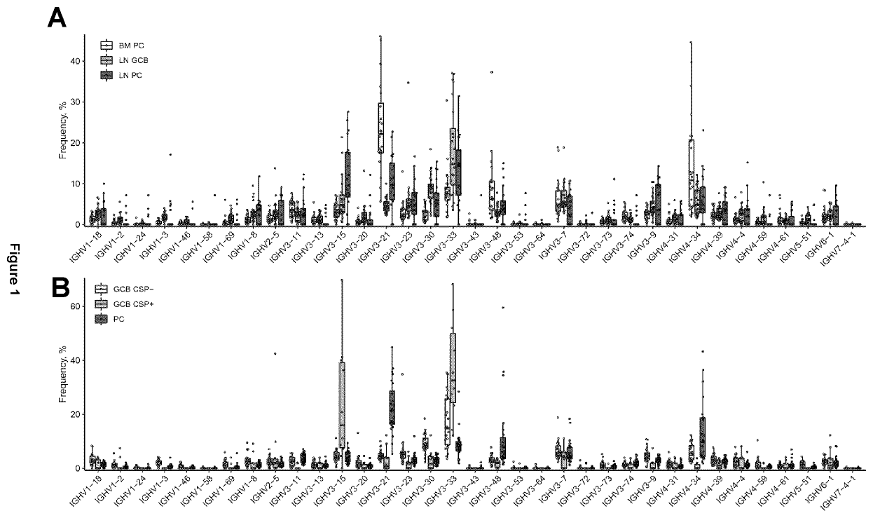

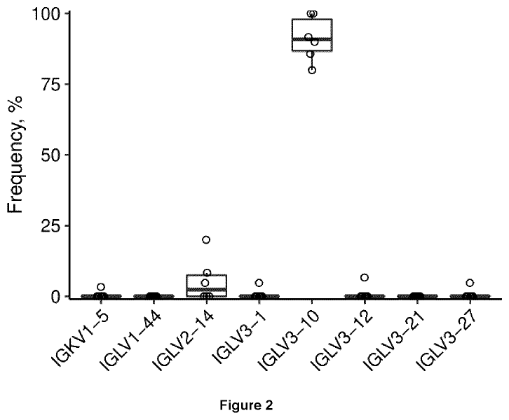

In mice transgenic for the human Ig antibody repertoire, B cells with signature IGHV3-33-encoded human antibodies against the PfCSP repeat were elicited in response to most immunogens. mAbs derived from these experiments were generally of high affinity to the repeat. For immunogens that contained the PfCSP cross-reactive epitopes (NPDP, NVDP motifs) and efficient T-cell help (PADRE epitope), high affinity to junction peptides was observed, and was paralleled with high affinity to NANP peptides. Unexpectedly, we observed that/GHV3-15 expressing B cells were also recruited to germinal center reactions and differentiated into plasma cells in lymph nodes (FIG. 1A). The enrichment of IGHV3-15 expressing cells was even more pronounced among CSP+ germinal center (GC) B cells compared to CSP− GC B cells, confirming that these gene segments were used by B cells that actively participated in the anti-immunogen response (FIG. 1B). Nearly all of these cells had IGLV3-10 encoded light chains (FIG. 2).

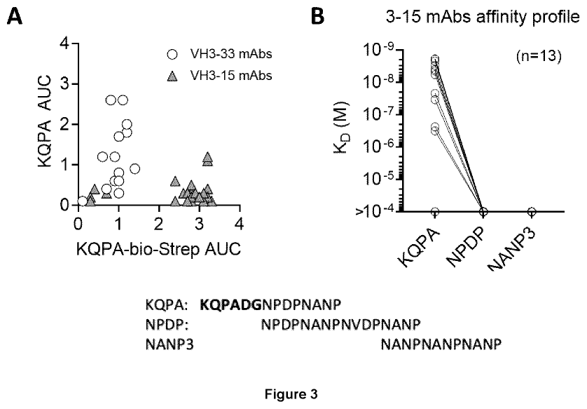

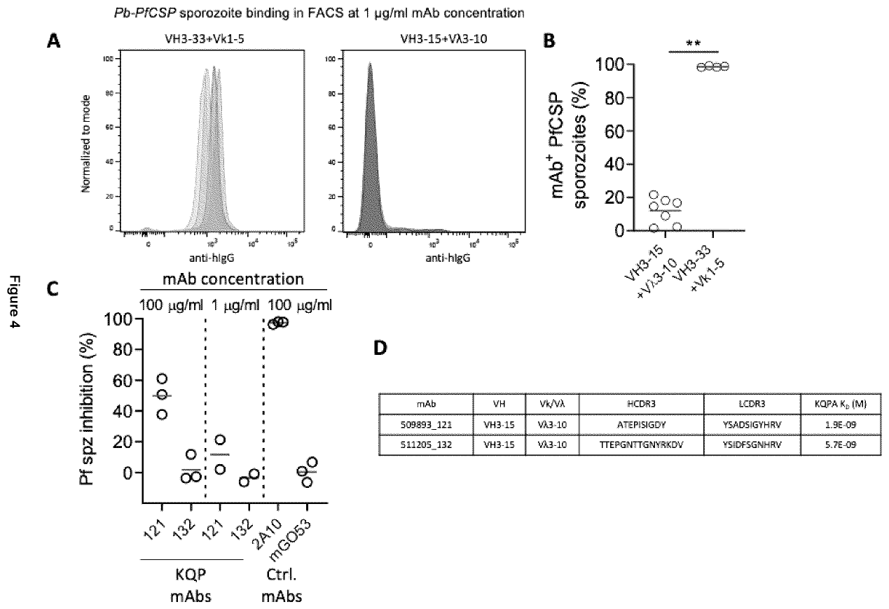

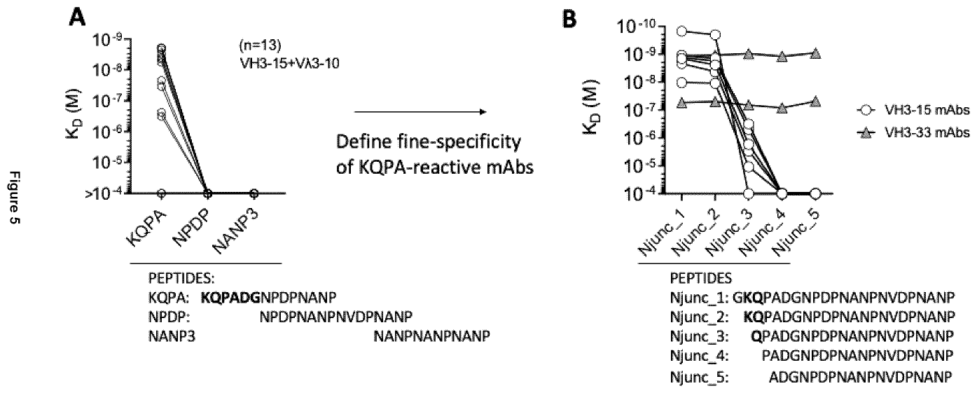

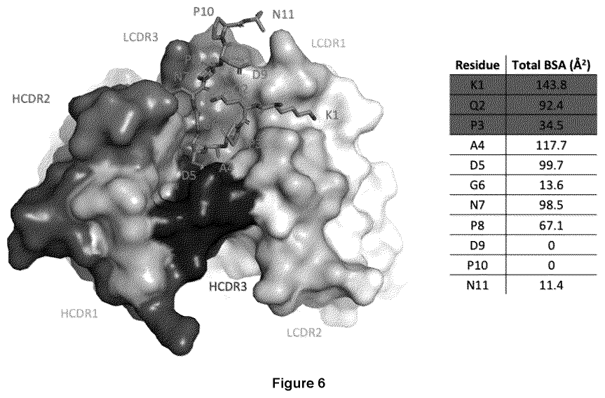

Binding of VH3-15/VL3-10 mAbs to KQPA peptide in ELISA was detected when biotinylated peptide was captured onto a Streptavidin-pre-coated plate, but not when KQPA peptide was directly coated onto high-binding plates (FIG. 3). Thus, direct immobilization of the KQPA peptide to the plates masks specific residues in the KQPA peptide that are recognized by VH3-15/VL3-10 mAbs. In contrast, VH3-33/VK1-5 mAbs showed binding only when the KQPA peptide is directly coated to the plate indicating the cross-reactivity of the mAbs to the minor repeat unit NPDP in the peptide. We independently verified the binding profiles of mAbs by measuring their affinity to the target peptides using Surface Plasmon Resonance. From several mAbs tested, VH3-15/VL3-10 mAbs were found to be KQPA-peptide specific with up to nanomolar affinity (FIG. 3) but did not bind to sporozoites in contrast to VH3-33/VK1-5 mAbs (FIG. 4). Two representative high affinity VH3-15/VL3-10 mAbs (mAb 509893_121 and mAb 511205_132) showed low hepatocyte Pf traversal inhibition in vitro (FIG. 4). Next, various peptides were screened to better understand the specificity of VH3-15/VL3-10 antibodies. VH3-15 mAbs were found to recognize a few amino acids in the PfCSP N-terminal Region I/junction with high affinity. Removal of the KQ motif in the PfCSP N-terminal junction abrogated mAb binding by these VH3-15/VL3-10 mAbs (FIG. 5). The crystal structure of Fab 509893_21 (VH3-15/VL3-10) was solved in complex with a KQPA-containing peptide. The structure confirmed the KQ residues were extensively contacted by this VH3-15/VL3-10 antibody (FIG. 6). The three residues KQP combined contributed 40% of total peptide interactions (FIG. 6).

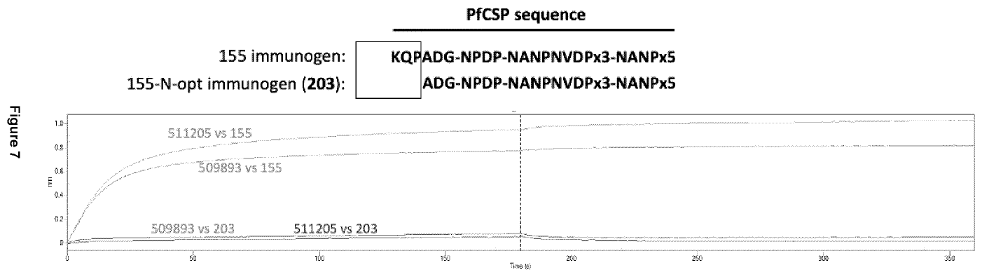

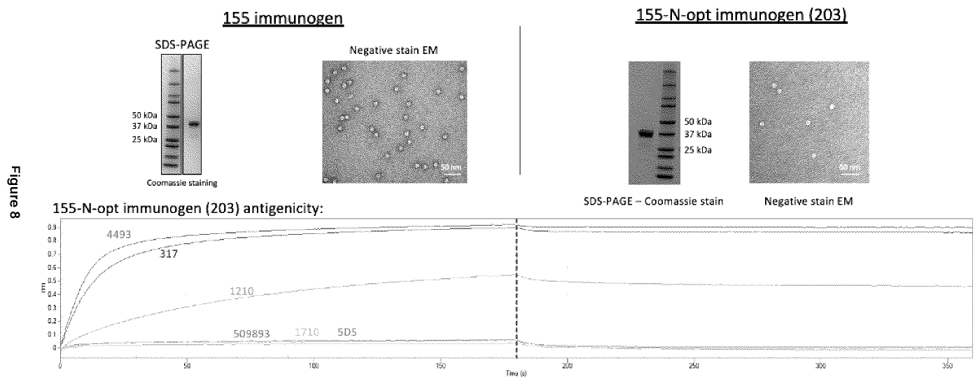

This data suggested that KQ, and/or more substantially KQP, residues could be removed in the immunogens to avoid the induction of B cell responses dominated by cells expressing high-affinity VH3-15 mAbs, which are non-sporozoite reactive and non-inhibitory as described. Refinement of the immunogen N terminus was undertaken using different strategies: e.g. deletion of KQP residues to start immunogen with PfCSP “ADG” sequence or with PfCSP “NPDP” sequence, and/or new signal peptide secretion sequence (e.g. derived from Human IgKVIII: MDMRVPAQLLGLLLLWLRGARC). Such immunogens (e.g. 203) abolished binding by the low parasite-inhibitory VH3-15/VL3-10 mAbs, in contrast to the previous immunogens that contained the KQP residues (e.g. 155) which bound with high affinity to the non-/low parasite-inhibitory VH3-15/VL3-10 mAbs (FIGS. 7 and 8). Like previous immunogens (e.g. 155), these 155-N-opt immunogens (e.g. 203) expressed well and showed favorable biophysical characteristics: they were pure and well-assembled nanoparticles, bound with high affinity to potently inhibitory junction/repeat mAbs (4493 and 317), bound with intermediate affinity to a mAb with modest inhibitory potential (1210), and did not bind to non-inhibitory mAbs or mAbs with low inhibitory potential (1710, 5D5 and 509893_21) (FIG. 8).

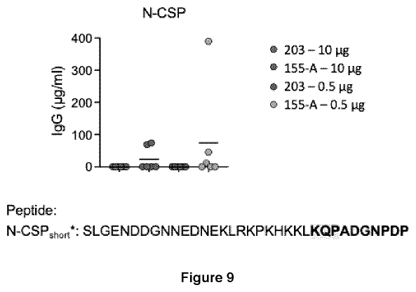

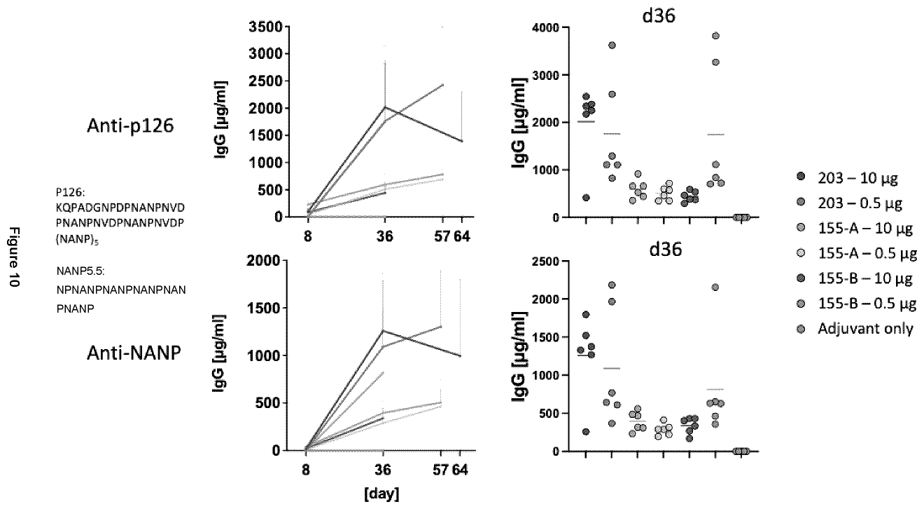

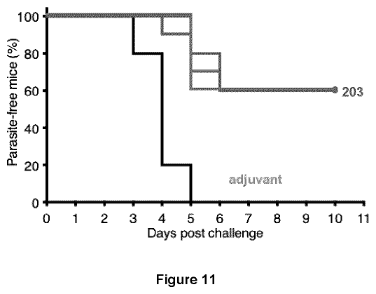

Mice transgenic for the human Ig antibody repertoire (e.g. Kymice) were immunized with these 155-N-opt immunogens (e.g. 203). In contrast to the previous immunogens that contained the KQP residues (e.g. 155), these new immunogens did not induce responses against the non-protective N-terminal junction KQP epitope (FIG. 9). Surprisingly, a much stronger anti-PfCSP response was observed in mice of human immunoglobulin repertoire immunized with 155-N-opt immunogens (e.g. 203) compared to the previous immunogens that contained the KQP residues (e.g. 155) (FIG. 10). We confirmed that the strong immune response elicited by 155-N-opt immunogens (e.g. 203) was indeed protective against infection with Pb-PfCSP transgenic parasites in a mosquito-bite challenge model in WT mice (FIG. 11). Together, these unexpected findings point towards the superiority of a PfCSP immunogen lacking these Lys and/or Gln and/or Pro amino acids to drive the most efficient response to the highly-inhibitory PfCSP junction/repeat epitopes in the context of a human immunoglobulin repertoire.

### Example 2

The immunogenicity of high-mannose glycosylated (203F+Kif) or non-glycosylated (211F) immunogens, both without KQP residues were tested in mice. WT mice (n=5 per group) were immunized i.m. with the immunogens at two doses (0.5 μg or 10 μg/mouse) in adjuvant (LMQ, TLR4 stimulating) or with adjuvant alone. The 203 and 211 sequences are as described herein and methods are as described above in Example 1. Briefly, serum IgG responses against the indicated peptides were measured by ELISA at different time points after the first (d0) or second (d28) immunization. Data show the means of means with SD from three independent experiments with 5 mice per group.

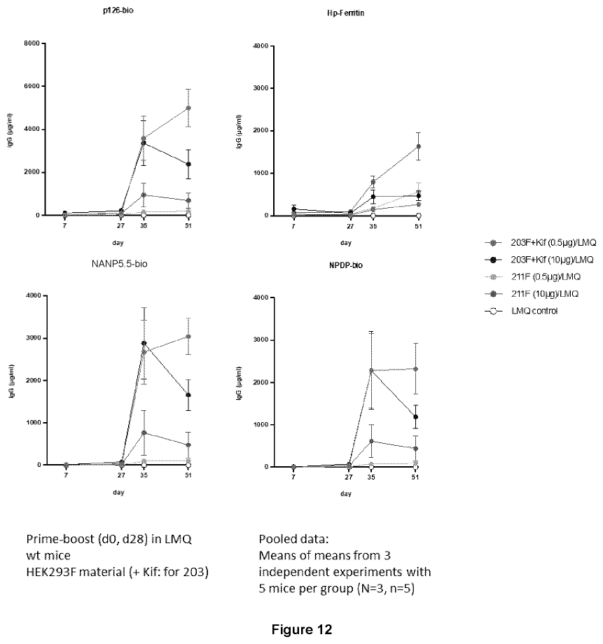

FIG. 12 shows that glycosylation increased immunogenicity against amino acid sequences representing the N-terminal junction and NANP repeat domain (P126-bio; top left), the NANP repeat domain (NANP5.5-bio; bottom left), and the NPDP domain (NPDP-bio; bottom right) but not against the Hp-ferritin backbone (Hp-ferritin; top right). This suggests that the increase in IgG serum response is specific for the malaria immunogen.

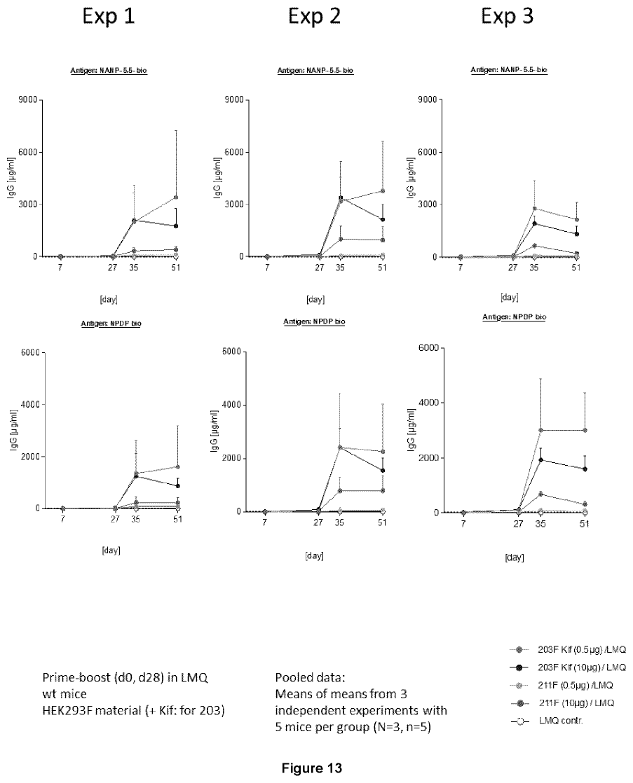

FIG. 13 similarly shows that glycosylation reproducibly increased immunogenicity against the NANP repeat domain (NANP5.5-bio; top), and the NPDP domain (NPDP-bio; bottom).

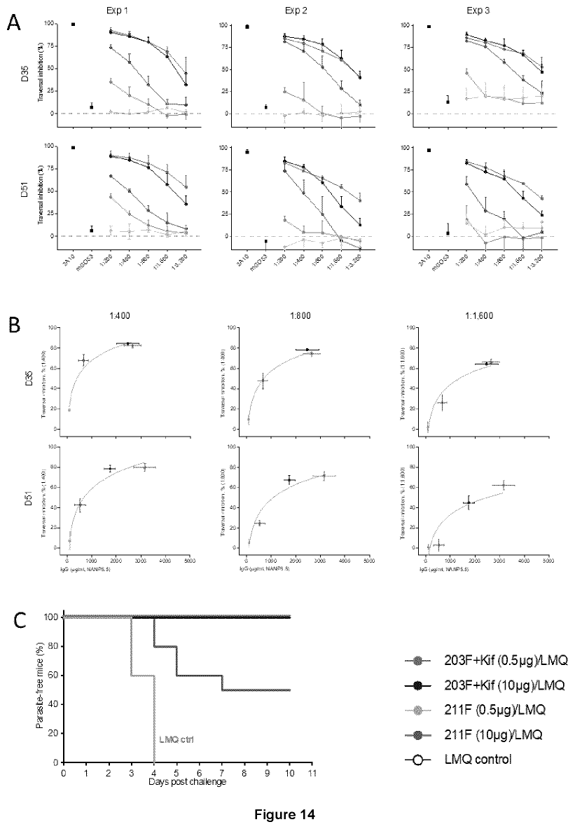

Next, sporozoite traversal inhibition assays, as described above in Example 1, were carried out using the high-mannose glycosylated (203F+Kif) or non-glycosylated (211F) immunogens, both without KQP residues. FIGS. 14A and 14B show that the percent traversal inhibition was higher with the glycosylated immunogen as both doses tested, as compared to the non-glycosylated immunogen.

FIG. 14C shows that both doses of the glycosylated immunogen protected 100% of mice from parasite infection 10 days after exposure to bites of three infectious mosquitos on day 52 after immunization. The non-glycosylated immunogen protected 50% of mice and the control provided no protection and all mice were infected by day 4 after exposure. These data indicate that, while omitting the KQP residues is protective against malaria infection, the combination of omitting the KQP residues and glycosylating the immunogen improves the protective effect.

### Example Sequences

153:
ETGKQPADGNPDPNANPNVDPNANPNVDPNANPNVDPNANPNANPNANPNANPNANPSG
GSGGSGGSGGSGGSDIIKLLNEQVNKEMQSSNLYMSMSSWCYTHSLDGAGLFLFDHAAEEYEHAK
KLIIFLNENNVPVQLTSISAPEHKFEGLTQIFQKAYEHEQHISESINNIVDHAIKSKDHATFNFLQWYVAE
QHEEEVLFKDILDKIELIGNENHGLYLADQYVKGIAKSRKSGGSASAKFVAAWTLKAAA
154:
ETGKQPADGNPDPNANPNVDPNANPNVDPNANPNVDPNANPNANPNANPNANPNANPNA
NPNANPNANPNANPNANPNANPNANPNANPNANPNANPNANPNANPNANPSGGSGGSGGSGGSG
GSDIIKLLNEQVNKEMQSSNLYMSMSSWCYTHSLDGAGLFLFDHAAEEYEHAKKLIIFLNENNVPVQL
TSISAPEHKFEGLTQIFQKAYEHEQHISESINNIVDHAIKSKDHATFNFLQWYVAEQHEEEVLFKDILDKI
ELIGNENHGLYLADQYVKGIAKSRKSGGSASAKFVAAWTLKAAA
155:
ETGKQPADGNPDPNANPNVDPNANPNVDPNANPNVDPNANPNANPNANPNANPNANPSGGSGGS
GGSGGSGGSDIIKLLNEQVNKEMQSSNLYMSMSSWCYTHSLDGAGLFLFDHAAEEYEHAKKLIIFLN
ENNVPVQLTSISAPEHNFTGLTQIFQKAYEHEQHISNSTNNIVDHAIKSKDHATFNFLQWYVAEQHEEE
VLFKDILDKIELIGNENHGLYLADQYVKGIAKSRKSGGSASAKFVAAWTLKAAA
156:
ETGKQPADGNPDPNANPNVDPNANPNVDPNANPNVDPNANPNANPNANPNANPNANPNA
NPNANPNANPNANPNANPNANPNANPNANPNANPNANPNANPNANPNANPSGGSGGSGGSGGSG
GSDIIKLLNEQVNKEMQSSNLYMSMSSWCYTHSLDGAGLFLFDHAAEEYEHAKKLIIFLNENNVPVQL
TSISAPEHNFTGLTQIFQKAYEHEQHISNSTNNIVDHAIKSKDHATFNFLQWYVAEQHEEEVLFKDILDK
IELIGNENHGLYLADQYVKGIAKSRKSGGSASAKFVAAWTLKAAA
203:
ADGNPDPNANPNVDPNANPNVDPNANPNVDPNANPNANPNANPNANPNANPSGGSGGSG
GSGGSGGSDIIKLLNEQVNKEMQSSNLYMSMSSWCYTHSLDGAGLFLFDHAAEEYEHAKKLIIFLNEN
NVPVQLTSISAPEHNFTGLTQIFQKAYEHEQHISNSTNNIVDHAIKSKDHATFNFLQWYVAEQHEEEVL
FKDILDKIELIGNENHGLYLADQYVKGIAKSRKSGGSASAKFVAAWTLKAAA
205:
ADGNPDPNANPNVDPNANPNVDPNANPNVDPNANPNANPNANPNANPNANPNANPNANP
NANPNANPNANPNANPNANPNANPNANPNANPNANPNANPNANPSGGSGGSGGSGGSGGSDIIKL
LNEQVNKEMQSSNLYMSMSSWCYTHSLDGAGLFLFDHAAEEYEHAKKLIIFLNENNVPVQLTSISAPE
HNFTGLTQIFQKAYEHEQHISNSTNNIVDHAIKSKDHATFNFLQWYVAEQHEEEVLFKDILDKIELIGNE
NHGLYLADQYVKGIAKSRKSGGSASAKFVAAWTLKAAA
206:
ADGNPDPNANPNVDPNANPNVDPNANPNVDPNANPNANPNANPNANPNANPNANPNANP
NANPNANPNANPNANPNANPNANPNANPNANPNANPNANPNANPSGGSGGSGGSGGSGGSDIIKL
LNEQVNKEMQSSNLYMSMSSWCYTHSLDGAGLFLFDHAAEEYEHAKKLIIFLNENNVPVQLTSISAPE
HKFEGLTQIFQKAYEHEQHISESINNIVDHAIKSKDHATFNFLQWYVAEQHEEEVLFKDILDKIELIGNE
NHGLYLADQYVKGIAKSRKSGGSASAKFVAAWTLKAAA
207:
ADGNPDPNANPNVDPNANPNVDPNANPNVDPNANPNANPNANPNANPNANPSGGSGGSG
GSGGSGGSDIIKLLNEQVNKEMQSSNLYMSMSSWCYTHSLDGAGLFLFDHAAEEYEHAKKLIIFLNEN
NVPVQLTSISAPEHNFTGLTQIFQKAYEHEQHISNSTNNIVDHAIKSKDHATFNFLQWYVAEQHEEEVL
FKDILDKIELIGNENHGLYLADQYVKGIAKSRKS
208:
ADGNPDPNANPNVDPNANPNVDPNANPNVDPNANPNANPNANPNANPNANPNANPNANP
NANPNANPNANPNANPNANPNANPNANPNANPNANPNANPNANPSGGSGGSGGSGGSGGSDIIKL
LNEQVNKEMQSSNLYMSMSSWCYTHSLDGAGLFLFDHAAEEYEHAKKLIIFLNENNVPVQLTSISAPE
HNFTGLTQIFQKAYEHEQHISNSTNNIVDHAIKSKDHATFNFLQWYVAEQHEEEVLFKDILDKIELIGNE
NHGLYLADQYVKGIAKSRKS
209:
ADGNPDPNANPNVDPNANPNVDPNANPNVDPNANPNANPNANPNANPNANPSGGSGGSG
GSGGSGGSDIIKLLNEQVNKEMQSSNLYMSMSSWCYTHSLDGAGLFLFDHAAEEYEHAKKLIIFLNEN
NVPVQLTSISAPEHKFEGLTQIFQKAYEHEQHISESINNIVDHAIKSKDHATFNFLQWYVAEQHEEEVL
FKDILDKIELIGNENHGLYLADQYVKGIAKSRKS
210:
ADGNPDPNANPNVDPNANPNVDPNANPNVDPNANPNANPNANPNANPNANPNANPNANP
NANPNANPNANPNANPNANPNANPNANPNANPNANPNANPNANPSGGSGGSGGSGGSGGSDIIKL
LNEQVNKEMQSSNLYMSMSSWCYTHSLDGAGLFLFDHAAEEYEHAKKLIIFLNENNVPVQLTSISAPE
HKFEGLTQIFQKAYEHEQHISESINNIVDHAIKSKDHATFNFLQWYVAEQHEEEVLFKDILDKIELIGNE
NHGLYLADQYVKGIAKSRKS
211:
ADGNPDPNANPNVDPNANPNVDPNANPNVDPNANPNANPNANPNANPNANPSGGSGGSG
GSGGSGGSDIIKLLNEQVNKEMQSSNLYMSMSSWCYTHSLDGAGLFLFDHAAEEYEHAKKLIIFLNEN
NVPVQLTSISAPEHKFEGLTQIFQKAYEHEQHISESINNIVDHAIKSKDHATFNFLQWYVAEQHEEEVL
FKDILDKIELIGNENHGLYLADQYVKGIAKSRKSGGSASAKFVAAWTLKAAA
212:
ADGNPDPNANPNVDPNANPNVDPNANPNVDPNANPNANPNANPNANPNANPGGSGGSGG
SGGSGGSDIIKLLNEQVNKEMQSSNLYMSMSSWCYTHSLDGAGLFLFDHAAEEYEHAKKLIIFLNENN
VPVQLTSISAPEHKFEGLTQIFQKAYEHEQHISESINNIVDHAIKSKDHATFNFLQWYVAEQHEEEVLFK
DILDKIELIGNENHGLYLADQYVKGIAKSRKSGGSASAKFVAAWTLKAAA
213:
ADGNPDPNANPNVDPNANPNVDPNANPNVDPNANPNANPNANPNANPNANPGGSGGSGG
SGGSGGSDIIKLLNEQVNKEMQSSNLYMSMSSWCYTHSLDGAGLFLFDHAAEEYEHAKKLIIFLNENN
VPVQLTSISAPEHNFTGLTQIFQKAYEHEQHISNSTNNIVDHAIKSKDHATFNFLQWYVAEQHEEEVLF
KDILDKIELIGNENHGLYLADQYVKGIAKSRKSGGSASAKFVAAWTLKAAA

### REFERENCES

1. World Health Organization. World malaria report 2020. S. I.: WORLD HEALTH ORGANIZATION; 2020.
2. Toe K H, Jones CM, N'Fale S, Ismail H M, Dabire R K, Ranson H. Increased Pyrethroid Resistance in Malaria Vectors and Decreased Bed Net Effectiveness, Burkina Faso. Emerg Infect Dis. 2014 October; 20(10):1691-6.
3. Hamilton W L, Amato R, Pluijm R W van der, Jacob C G, Quang H H, Thuy-Nhien N T, et al. Evolution and expansion of multidrug-resistant malaria in southeast Asia: a genomic epidemiology study. The Lancet Infectious Diseases. 2019 Sep. 1; 19(9):943-51.
4. Hall B F, Fauci A S. Malaria Control, Elimination, and Eradication: The Role of the Evolving Biomedical Research Agenda. J INFECT DIS. 2009 December; 200(11):1639-43.
5. Cerami C, Frevert U, Sinnis P, Takacs B, Clavijo P, Santos M J, et al. The basolateral domain of the hepatocyte plasma membrane bears receptors for the circumsporozoite protein of Plasmodium falciparum sporozoites. Cell. 1992 Sep. 18; 70(6):1021-33.
6. Frevert U, Sinnis P, Cerami C, Shreffler W, Takacs B, Nussenzweig V. Malaria circumsporozoite protein binds to heparan sulfate proteoglycans associated with the surface membrane of hepatocytes. Journal of Experimental Medicine. 1993 May 1; 177(5):1287-98.
7. Menard R, Sultan A A, Cortes C, Altszuler R, van Dijk M R, Janse C J, et al. Circumsporozoite protein is required for development of malaria sporozoites in mosquitoes. Nature. 1997 Jan. 23; 385(6614):336-40.
8. Dame J B, Williams J L, McCutchan T F, Weber J L, Wirtz R A, Hockmeyer W T, et al. Structure of the gene encoding the immunodominant surface antigen on the sporozoite of the human malaria parasite Plasmodium falciparum. Science. 1984 Aug. 10; 225(4662):593-9.
9. Enea V, Ellis J, Zavala F, Arnot D E, Asavanich A, Masuda A, et al. DNA cloning of Plasmodium falciparum circumsporozoite gene: amino acid sequence of repetitive epitope. Science. 1984 Aug. 10; 225(4662):628-30.
10. Escalante A A, Grebert H M, Isea R, Goldman I F, Basco L, Magris M, et al. A study of genetic diversity in the gene encoding the circumsporozoite protein (CSP) of Plasmodium falciparum from different transmission areas—XVI. Asembo Bay Cohort Project. Mol Biochem Parasitol. 2002 December; 125(1-2):83-90.
11. Bailey J A, Mvalo T, Aragam N, Weiser M, Congdon S, Kamwendo D, et al. Use of massively parallel pyrosequencing to evaluate the diversity of and selection on Plasmodium falciparum csp T-cell epitopes in Lilongwe, Malawi. J Infect Dis. 2012 Aug. 15; 206(4):580-7.
12. Gaudinski M R, Berkowitz N M, Idris A H, Coates E E, Holman L A, Mendoza F, et al. A Monoclonal Antibody for Malaria Prevention. N Engl J Med. 2021 Aug. 26; 385(9):803-14.
13. Julien J P, Wardemann H. Antibodies against Plasmodium falciparum malaria at the molecular level. Nat Rev Immunol. 2019 Aug. 28; 19: 761-775.
14. Tan J, Sack B K, Oyen D, Zenklusen I, Piccoli L, Barbieri S, et al. A public antibody lineage that potently inhibits malaria infection through dual binding to the circumsporozoite protein. Nat Med. 2018 April; 24(4):401-7.
15. Kisalu N K, Idris A H, Weidle C, Flores-Garcia Y, Flynn B J, Sack B K, et al. A human monoclonal antibody prevents malaria infection by targeting a new site of vulnerability on the parasite. Nat Med. 2018 May; 24(4):408-16.
16. Kucharska I, Thai E, Srivastava A, Rubinstein J L, Pomes R, Julien J-P. Structural ordering of the Plasmodium berghei circumsporozoite protein repeats by inhibitory antibody 3D11. eLife. 2020 Nov. 30; 9:e59018.
17. Kucharska I, Hossain L, Ivanochko D, Yang Q, Rubinstein J L, Pomes R, et al. Structural basis of Plasmodium vivax inhibition by antibodies binding to the circumsporozoite protein repeats. eLife. 2022 Jan. 13; 11:e72908.
18. Murugan R, Buchauer L, Triller G, Kreschel C, Costa G, Pidelaserra Marti G, et al. Clonal selection drives protective memory B cell responses in controlled human malaria infection. Sci Immunol. 2018 Feb. 16; 3(20):eaap8029.
19. Murugan R, Scally S W, Costa G, Mustafa G, Thai E, Decker T, et al. Evolution of protective human antibodies against Plasmodium falciparum circumsporozoite protein repeat motifs. Nat Med. 2020 May 25; 26(7):1135-45.
20. Scally S W, Murugan R, Bosch A, Triller G, Costa G, Mordmuller B, et al. Rare PfCSP C-terminal antibodies induced by live sporozoite vaccination are ineffective against malaria infection. J Exp Med. 2018 Jan. 2; 215(1):63-75.
21. Thai E, Costa G, Weyrich A, Murugan R, Oyen D, Flores-Garcia Y, Prieto K, Bosch A, Valleriani A, Wu N C, Pholcharee T, Scally S W, Wilson I A, Wardemann H, Julien J P, Levashina E A. A high-affinity antibody against the CSP N-terminal domain lacks Plasmodium falciparum inhibitory activity. J Exp Med. 2020 Aug. 13; 217 (11): e20200061.
22. A Phase 3 Trial of RTS,S/AS01 Malaria Vaccine in African Infants. N. Engl. J. Med. 367, 2284-2295 (2012).
23. M. S. Datoo, M. H. Natama, A. Some, O. Traore, T. Rouamba, D. Bellamy, P. Yameogo, D. Valia, M. Tegneri, F. Ouedraogo, R. Soma, S. Sawadogo, F. Sorgho, K. Derra, E. Rouamba, B. Orindi, F. Ramos Lopez, A. Flaxman, F. Cappuccini, R. Kailath, S. Elias, E. Mukhopadhyay, A. Noe, M. Cairns, A. Lawrie, R. Roberts, I. Valea, H. Sorgho, N. Williams, G. Glenn, L. Fries, J. Reimer, K. J. Ewer, U. Shaligram, A. V. S. Hill, H. Tinto, Efficacy of a low-dose candidate malaria vaccine, R21 in adjuvant Matrix-M, with seasonal administration to children in Burkina Faso: a randomised controlled trial. Lancet. 397, 1809-1818 (2021).
24. Triller G, Scally S W, Costa G, Pissarev M, Kreschel C, Bosch A, Marois E, Sack B K, Murugan R, Salman A M, Janse C J, Khan S M, Kappe S H I, Adegnika A A, Mordmuller B, Levashina E A, Julien J P, Wardemann H. Natural Parasite Exposure Induces Protective Human Anti-Malarial Antibodies. Immunity. 2017 Dec. 19; 47(6):1197-1209.e10.
25. Sattabongkot J, Yimamnuaychoke N, Leelaudomlipi S, Rasameesoraj M, Jenwithisuk R, Coleman R E, Udomsangpetch R, Cui L, Brewer T G. Establishment of a human hepatocyte line that supports in vitro development of the exo-erythrocytic stages of the malaria parasites Plasmodium falciparum and P. vivax. Am J Trop Med Hyg. 2006 May; 74(5):708-15.
26. Wardemann H, Yurasov S, Schaefer A, Young J W, Meffre E, Nussenzweig M C. Predominant autoantibody production by early human B cell precursors. Science. 2003 Sep. 5:301(5638):1374-7.

What is claimed is:

1. A malarial immunogen or a variant thereof comprising at least a portion of the wild-type PfCSP amino acid sequence lacking a KQ motif.

2. The malarial immunogen of claim 1, lacking a KQP motif.

3. The malarial immunogen of claim 1 or claim 2, lacking an N-terminal ETG motif.

4. The malarial immunogen of any one of claims 1 to 3, comprising the following motifs:
NPDPₐ; PDPNₐ; DPNPₐ; or PNPDₐ;
NANP_{b}; ANPN_{b}; NPNA_{b}; or PNAN_{b};
NVDP_{c}; VDPN_{c}; DPNV_{c}; or PNVD_{c}; and
NANP_{d}; ANPN_{d}; NPNA_{d}; or PNAN_{d}.
wherein a, b, c, and d are each independently 0 or greater and wherein a+b+c+d is at least 2.

5. The malarial immunogen of claim 4, wherein a, b, c, d or any combination thereof are each independently at least about 1.

6. The malarial immunogen of claim 5, wherein a, b, c, d or any combination thereof are each independently from about 1 to about 40.

7. The malarial immunogen of claim 6, wherein a, b, c, and d are each independently from about 1 to about 100, such as from about 1, about 2, about 3, about 4, about 5, about 6, about 7, about 8, about 9, about 10, about 11, about 12, about 13, about 14, about 15, about 16, about 17, about 18, about 19, or about 20 to about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 60, about 70, about 75, about 80, about 90, or about 100, such as from about 1 to about 40, or about 1 to about 20, or from 1 to about 10.

8. The malarial immunogen of any one of claims 4 to 7, wherein a is 1.

9. The malarial immunogen of any one of claims 4 to 8, wherein b is 1.

10. The malarial immunogen of any one of claims 4 to 9, wherein c is 3.

11. The malarial immunogen of any one of claims 4 to 10, wherein d is 5.

12. The malarial immunogen of any one of claims 4 to 11, wherein d is 18.5.

13. The malarial immunogen of any one of claims 4 to 12, wherein when a, b, c, and/or d are greater than 1 such that the respective motif is at least partially repeated, the repeated motifs are each independently contiguous.

14. The malarial immunogen of any one of claims 4 to 13, wherein when a, b, c, and/or d are greater than 1 such that the respective motif is at least partially repeated, the repeated motifs are each independently non-contiguous.

15. The malarial immunogen of any one of claims 4 to 14, wherein the motifs are in the order NPDPₐ-NANP_{b}-NVDP_{c}-NANP_{d}.

16. The malarial immunogen of any one of claims 1 to 15, further comprising an N-terminal ADG or PADG motif, wherein the ADG or PADG motif optionally repeats alone or in combination with at least one of the NPDPₐ, NANP_{b}, NVDP_{c}, and NANP_{d} motifs.

17. The malarial immunogen of any one of claims 1 to 16, wherein the variant comprises at least about 70%, 75%, 80%, 85%, 90%, 95%, 97%, or 99% sequence identity to the wild-type PfCSP amino acid sequence lacking the KQ motif.

18. The malarial immunogen of any one of claims 1 to 17, wherein the malarial immunogen is fused directly or indirectly to a nanocage monomer peptide.

19. The malarial immunogen of claim 18, wherein the nanocage monomer is ferritin, apoferritin, encapsulin, SOR, lumazine synthase, pyruvate dehydrogenase, carboxysome, vault proteins, GroEL, heat shock protein, E2P, or MS2 coat protein, or fragments thereof, or variants thereof.

20. The malarial immunogen of claim 19, wherein the nanocage monomer is provided as two or more self-assembling subunits.

21. The malarial immunogen of any one of claims 18 to 20, wherein the nanocage monomer peptide is from Helicobacter pylori.

22. The malarial immunogen of any one of claims 18 to 20, wherein the nanocage monomer peptide is not human.

23. The malarial immunogen of any one of claims 18 to 22, wherein the nanocage monomer peptide is modified to reduce an anti-nanocage monomer peptide immune response.

24. The malarial immunogen of any one of claims 18 to 23, wherein the nanocage monomer peptide is modified to enhance the antigen immune response.

25. The malarial immunogen of claim 23 or 24, wherein the nanocage monomer peptide is at least partially or fully covered.

26. The malarial immunogen of claim 25, wherein the nanocage monomer peptide is at least partially glycan covered.

27. The malarial immunogen of claim 26, wherein the nanocage monomer peptide is fully glycan covered.

28. The malarial immunogen of any one of claims 21 to 27, wherein the nanocage monomer comprises at least one NXT and/or NXS glycosylation motif.

29. The malarial immunogen of any one of claims 21 to 27, wherein the nanocage monomer is glycosylated at N79 and/or N99.

30. The malarial immunogen of any one of claims 21 to 29, wherein the nanocage monomer is glycosylated with high-mannose glycans.

31. The malarial immunogen of any one of claims 18 to 30, wherein a plurality of the nanocage monomer peptides self-assemble into a nanocage.

32. The malarial immunogen of claim 31, wherein the immunogenic peptide decorates the interior and/or exterior surface of the nanocage.

33. The malarial immunogen of any one of claims 1 to 32, further comprising a peptide that provides exogenous T cell help and/or a peptide that provides autologous T cell help.

34. The malarial immunogen of claim 33, wherein the peptide that provides exogenous T cell help comprises a PADRE peptide and/or a peptide derived from a pathogenic molecule, such as a tetanus toxoid peptide.

35. The malarial immunogen of claim 34, wherein the PADRE peptide comprises the amino acid sequence AKFVAAWTLKAAA, or a functional variant thereof having at least 70% sequence identity thereto or a fragment of either thereof.

36. The malarial immunogen of any one of claims 33 to 35, wherein the peptide that provides autologous T cell help comprises a PfCSP T cell peptide epitope.

37. The malarial immunogen of any one of claims 33 to 36, wherein the peptide that provides exogenous T cell help and/or the peptide that provides autologous T cell help independently decorates the interior and/or exterior surface of the assembled nanocage.

38. The malarial immunogen of any one of claims 1 to 37, further comprising a linker between any one or more of the motifs, the nanocage monomer, and any further peptides, such as the peptide that provides exogenous T cell help and/or the peptide that provide autologous T cell help.

39. The malarial immunogen of claim 38, wherein the linker is a GGS linker.

40. The malarial immunogen of claim 39, wherein the linker comprises the amino acid sequence:
GGS;
GGGGSGGSGGSGGS;
and/or
GGGGGSGGSGGSGGS.

41. The malarial immunogen of any one of claims 1 to 40, comprising or consisting of the sequence:
ADG-NPDP-NANPNVDP3-NANP5-Hpferr-PADRE;
ADG-NPDP-NANPNVDP3-NANP18-Hpferr-PADRE;
ADG-NPDP-NANPNVDP3-NANP18.5-Hpferr-PADRE;
ADG-NPDP-NANPNVDP3-NANP5-LS-PADRE; and/or
ADG-NPDP-NANPNVDP3-NANP18-LS-PADRE;
ADG-NPDP-NANPNVDP3-NANP18.5-LS-PADRE.

42. The malarial immunogen of any one of claims 1 to 41, comprising any one of the following amino acid sequences:
203:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHNFTGLTQIF
QKAYEHEQHISNSTNNIVDHAIKSKDHATFNFLQWYVAEQHEEEVLFKDI
LDKIELIGNENHGLYLADQYVKGIAKSRKSGGSASAKFVAAWTLKAAA
205:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHNFTGLTQ
IFQKAYEHEQHISNSTNNIVDHAIKSKDHATFNFLQWYVAEQHEEEVLFK
DILDKIELIGNENHGLYLADQYVKGIAKSRKSGGSASAKFVAAWTLKAAA
206:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFK
DILDKIELIGNENHGLYLADQYVKGIAKSRKSGGSASAKFVAAWTLKAAA
207:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHNFTGLTQIF
QKAYEHEQHISNSTNNIVDHAIKSKDHATFNFLQWYVAEQHEEEVLFKDI
LDKIELIGNENHGLYLADQYVKGIAKSRKS
208:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHNFTGLTQ
IFQKAYEHEQHISNSTNNIVDHAIKSKDHATFNFLQWYVAEQHEEEVLFK
DILDKIELIGNENHGLYLADQYVKGIAKSRKS
209
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDI
LDKIELIGNENHGLYLADQYVKGIAKSRKS
210:
ADGNPDPNANPNVDPNANPNVDPNANPNVDPNANPNANPNANPNANPNAN
PNANPNANPNANPNANPNANPNANPNANPNANPNANPNANPNANPNANPN
ANPSGGSGGSGGSGGSGGSDIIKLLNEQVNKEMQSSNLYMSMSSWCYTHS
LDGAGLFLFDHAAEEYEHAKKLIIFLNENNVPVQLTSISAPEHKFEGLTQ
IFQKAYEHEQHISESINNIVDHAIKSKDHATFNFLQWYVAEQHEEEVLFK
DILDKIELIGNENHGLYLADQYVKGIAKSRKS
211:
ADGNPDPNANPNVDPNANPNVDPNANPNVDPNANPNANPNANPNANPNAN
PSGGSGGSGGSGGSGGSDIIKLLNEQVNKEMQSSNLYMSMSSWCYTHSLD
GAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDI
LDKIELIGNENHGLYLADQYVKGIAKSRKSGGSASAKFVAAWTLKAAA
212:
ADGNPDPNANPNVDPNANPNVDPNANPNVDPNANPNANPNANPNANPNAN
PGGSGGSGGSGGSGGSDIIKLLNEQVNKEMQSSNLYMSMSSWCYTHSLDG
AGLFLFDHAAEEYEHAKKLIIFLNENNVPVQLTSISAPEHKFEGLTQIFQ
KAYEHEQHISESINNIVDHAIKSKDHATFNFLQWYVAEQHEEEVLFKDIL
DKIELIGNENHGLYLADQYVKGIAKSRKSGGSASAKFVAAWTLKAAA
213:
ADGNPDPNANPNVDPNANPNVDPNANPNVDPNANPNANPNANPNANPNAN
PGGSGGSGGSGGSGGSDIIKLLNEQVNKEMQSSNLYMSMSSWCYTHSLDG
AGLFLFDHAAEEYEHAKKLIIFLNENNVPVQLTSISAPEHNFTGLTQIFQ
KAYEHEQHISNSTNNIVDHAIKSKDHATFNFLQWYVAEQHEEEVLFKDIL
DKIELIGNENHGLYLADQYVKGIAKSRKSGGSASAKFVAAWTLKAAA
or a functional variant having at least 70% sequence identity thereto, or a functional fragment of either thereof.

43. A nucleic acid molecule encoding the malarial immunogen of any one of claims 1 to 42.

44. A vector comprising the nucleic acid molecule of claim 43.

45. A host cell comprising the vector of claim 41 and producing the malarial immunogen of any one of claims 1 to 42.

46. A vaccine comprising the malarial immunogen of any one of claims 1 to 42.

47. The vaccine of claim 46, further comprising an adjuvant.

48. An antibody that binds to the malarial immunogen of any one of claims 1 to 47.

49. A method of treating and/or preventing malaria, comprising administering the immunogen, nucleotide, vector, cell, or vaccine of any one of claims 1 to 47.

50. Use of the immunogen, nucleotide, vector, cell, or vaccine of any one of claims 1 to 47 for treating and/or preventing malaria.

51. The immunogen, nucleotide, vector, cell, or vaccine of any one of claims 1 to 47 for use in treating and/or preventing malaria.

52. The method, use, or immunogen, nucleotide, vector, cell, or vaccine of any one of claims 49 to 51, wherein the preventative and/or treatment effect is boostable.

53. The method, use, or immunogen, nucleotide, vector, cell, or vaccine of any one of claims 49 to 51, wherein the preventative and/or treatment effect persists for at least about 6 months or more, such as about 9 months or more, about 12 months or more, about 1, about 2, about 3, about 4, about 5, about 6, about 7, about 8, about 9, or about 10 years or more.
